(19) United States
(12) Patent Application Publication
(10) Pub. No.: US 2008/0214533 A1
(43) Pub. Date: Sep. 4, 2008

(54) Cyclic Iminocarbamates And Their Use

(75) Inventors: Susanne Rohrig, Essen (DE); Jens Pohlmann, Basel (CH); Sabine Arndt, Dortmund (DE); Mario Jeske, Solingen (DE); Metin Akbaba, Ratingen (DE); Elisabeth Perzborn, Wuppertal (DE); Christoph Gerdes, Leverkusen (DE); Karl-Heinz Schlemmer, Wuppertal (DE); Arounarith Tuch, Lyon (FR); Mario Lobell, Wuppertal (DE); Peter Nell, Wuppertal (DE); Nils Burkhardt, Velbert (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, LEVERKUSEN (DE)

(21) Appl. No.: 11/792,108

(22) PCT Filed: Nov. 22, 2005
(86) PCT No.: PCT/EP2005/012465

§ 371 Date: Dec. 13, 2007

(30) Foreign Application Priority Data
Dec. 2, 2004 (DE) 102004058062.6

(51) Int. Cl. A61K 31/5355; C07D 401/14; C07D 413/14; A61K 31/4439; C07D 417/14; A61K 31/497; A61P 7/00

(52) U.S. Cl. 514/228.8; 544/405; 544/96; 546/269.7; 514/255.05; 514/342

(57) ABSTRACT

The invention relates to novel cyclic iminocarbamates of formula (I),

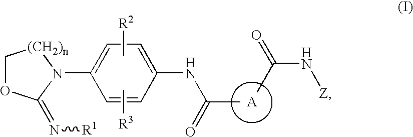

wherein R¹, R², R³, A, Z, and n have the meanings recited in the description, to a method for the production thereof, to pharmaceutical compositions containing such materials, and to methods for the treatment and/or prophylaxis of thromboembolic diseases using them.

The present application relates to novel cyclic iminocarbamates, to processes for their preparation, to their use for the treatment and/or prophylaxis of diseases and also to their use for preparing medicaments for the treatment and/or prophylaxis of diseases, in particular thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus haemostasis. In addition, thrombin is a potent effector of platelet aggregation which likewise contributes significantly to haemostasis.

Haemostasis is subject to a complex regulatory mechanism. Uncontrolled activation of the coagulant system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoagulability may—systemically—result in disseminated intravascular coagulation. Thromboembolic complications furthermore occur in microangiopathic haemolytic anaemias, extracorporeal blood circulation, such as haemodialysis, and also in connection with prosthetic heart valves.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favourable pharmacokinetic properties, preference is nowadays more and more given to low-molecular-weight heparin; however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is nonselective. Moreover, there is a high risk of bleeding; in particular, brain haemorrhages and gastrointestinal bleeding may occur, which may result in thrombopenia, drug-induced alopecia or osteoporosis [Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin K-dependent coagulation factors in the liver in a non-selective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Stürzebecher, Thrombosis Research 1999; 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77 (online publication August 2004)].

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors: Today and beyond” Curr. Opin. Investig. Drugs 2003, 4, 272-281; J. Ruef, H. A. Katus, “New anti-thrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Nonpeptidic low-molecular-weight factor Xa inhibitors are also described, for example, in WO 03/047520, WO 02/079145, WO 02/000651 and WO 02/000647.

It is an object of the present invention to provide novel substances for controlling disorders, in particular thromboembolic disorders.

The present invention provides compounds of the general formula (I)

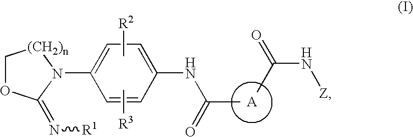

in which
n represents the number 1, 2 or 3,
R¹ represents hydrogen, (C₁-C₄)-alkyl, (C₁-C₄)-alkanoyl, cyano or hydroxyl,
R² and R³ are identical or different and independently of one another represent hydrogen, fluorine, chlorine, cyano, (C₁-C₃)-alkyl, cyclopropyl, trifluoromethyl, hydroxyl, (C₁-C₃)-alkoxy, trifluoromethoxy or amino,
A represents a phenylene or 5- or 6-membered heteroarylene ring where the two carboxamide groupings —CO—NH-phenyl and —CO—NH-Z are located at adjacent ring atoms of the phenylene or heteroarylene ring and phenylene and heteroarylene may additionally be substituted by halogen and/or (C₁-C₄)-alkyl,
and
Z represents phenyl, pyridyl, pyrimidinyl, pyrazinyl or thienyl, each of which may be mono- or disubstituted by identical or different substituents selected from the group consisting of fluorine, chlorine, cyano, (C₁-C₄)-alkyl (which for its part may be substituted by amino), ethynyl and amino,
   and its salts, solvates and solvates of the salts.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

In the context of the invention, (C₁-C₄)-alkyl and (C₁-C₃)-alkyl represent a straight-chain or branched alkyl radical having 1 to 4 and 1 to 3 carbon atoms, respectively. Preference is given to a straight-chain or branched alkyl radical having 1 to 3 carbon atoms. The following radicals may be mentioned by way of example and by way of preference: methyl, ethyl, n-propyl, isopropyl, n-butyl, isobutyl, sec-butyl and tert-butyl.

In the context of the invention, (C₁-C₃)-alkoxy represents a straight-chain or branched alkoxy radical having 1 to 3 carbon atoms. The following radicals may be mentioned by way of example and by way of preference: methoxy, ethoxy, n-propoxy and isopropoxy.

In the context of the invention, (C₁-C₄)-alkanoyl [(C₁-C₄)-acyl] represents a straight-chain or branched alkyl radical having 1 to 4 carbon atoms which carries a doubly attached oxygen atom in the 1-position and is attached via the 1-position. Preference is given to an alkanoyl radical having 2 or 3 carbon atoms. The following radicals may be mentioned by way of example and by way of preference: formyl, acetyl, propionyl, n-butyryl and isobutyryl.

In the context of the invention, halogen includes fluorine, chlorine, bromine and iodine. Preference is given to chlorine or fluorine.

In the context of the invention, 5- or 6-membered heteroarylene represents a bivalent monocyclic aromatic heterocycle (heteroaromatic) having 5 or 6 ring atoms in total and up to three identical or different ring heteroatoms from the group consisting of N, O and S, where the two carboxamide groupings independently of one another are each attached via a ring carbon atom or a ring nitrogen atom to heteroarylene. The following radicals may be mentioned by way of example: furylene, pyrrolylene, thienylene, pyrazolylene, imidazolylene, thiazolylene, oxazolylene, isoxazolylene, isothiazolylene, triazolylene, oxadiazolylene, thiadiazolylene, pyridylene, pyrimidinylene, pyridazinylene, pyrazinylene.

Preference is given to 5- or 6-membered heteroarylene groups having up to two heteroatoms from the group consisting of N, O and S, such as, for example, furylene, pyrrolylene, thienylene, thiazolylene, oxazolylene, imidazolylene, pyrazolylene, pyridylene, pyrimidinylene, pyridazinylene, pyrazinylene.

If radicals in the compounds according to the invention are substituted, the radicals can, unless specified otherwise, be mono- or polysubstituted. In the context of the present invention, the meanings of radicals which occur more than once are independent of one another. Substitution with one, two or three identical or different substituents is preferred.

Particular preference is given to substitution with one substituent.

A particular embodiment of the invention comprises compounds of the formula (I) in which
R¹ represents hydrogen, (C₁-C₄)-alkyl, (C₁-C₄)-alkanoyl or cyano.

Preference is given to compounds of the formula (I) in which
n represents the number 1 or 2,
R¹ represents hydrogen,
R² represents hydrogen and
R³ represents hydrogen, fluorine or methyl.

Preference is also given to compounds of the formula (I) in which
A represents a group of the formula

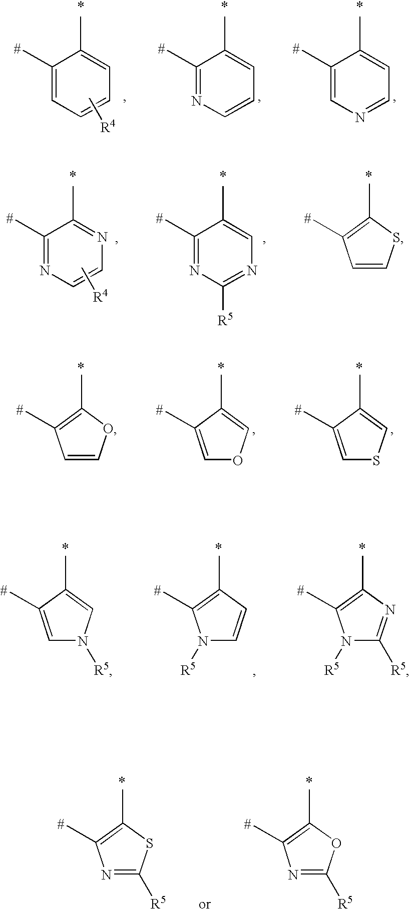

in which
R⁴ represents hydrogen, halogen or (C₁-C₄)-alkyl,
R⁵ represents hydrogen or (C₁-C₄)-alkyl
and
# and * represent the point of attachments to the —CO—NH-phenyl and the —CO—NH-Z grouping.

Preference is furthermore given to compounds of the formula (I) in which
A represents a group of the formula

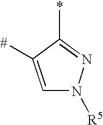

in which
R⁵ represents hydrogen or (C₁-C₄)-alkyl
and
# and * represent the point of attachments to the —CO—NH-phenyl and the —CO—NH-Z grouping.

Preference is also given to compounds of the formula (I) in which
Z represents a group of the formula

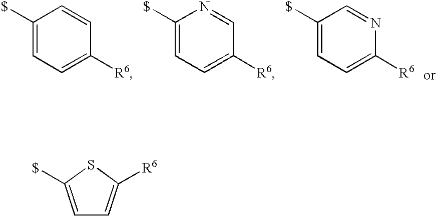

in which
R⁶ represents fluorine, chlorine, cyano, methyl or ethynyl,
and
$ represents the point of attachment to the nitrogen atom.

Particular preference is given to compounds of the formula (I) in which
n represents the number 1 or 2,
R¹ represents hydrogen,
R² represents hydrogen,
R³ represents hydrogen, fluorine or methyl,
A represents a group of the formula

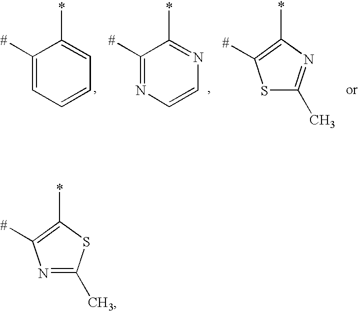

in which
# represents the point of attachment to the —CO—NH-phenyl grouping and
* represents the point of attachment to the —CO—NH-Z grouping,
and
Z represents a group of the formula

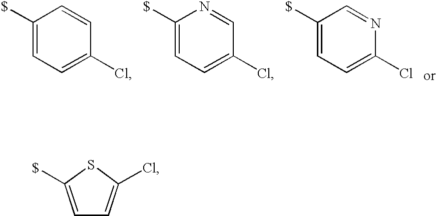

in which $ represents the point of attachment to the nitrogen atom,
   and its salts, solvates and solvates of the salts.

Here, in a particular embodiment of the invention,
A represents a group of the formula

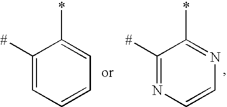

in which # and * represent the point of attachments to the —CO—NH-phenyl and the —CO—NH-Z grouping.

Particular preference is given to the compounds of the formula (I) of the following structures:

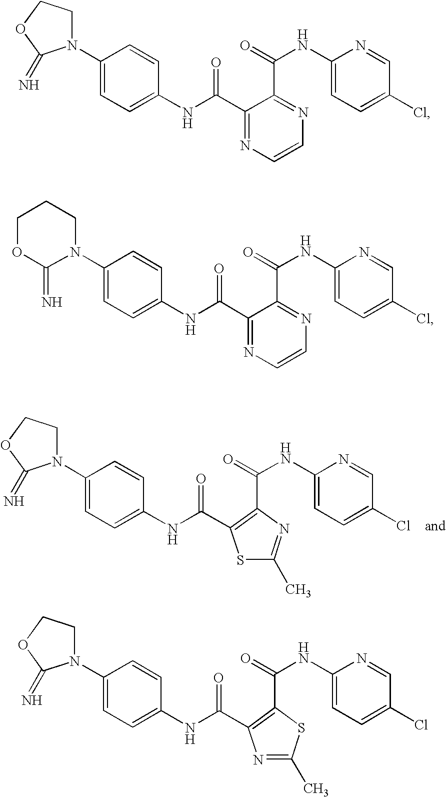

and their salts, solvates and solvates of the salts.

The individual radical definitions given in the respective combinations or preferred combinations of radicals are, independently of the particular given combinations of radicals, also replaced by any radical definitions of other combinations.

Very particular preference is given to combinations of two or more of the preferred ranges mentioned above.

The invention furthermore provides a process for preparing the compounds of the formula (I) according to the invention in which R¹ represents hydrogen, characterized in that compounds of the formula (II)

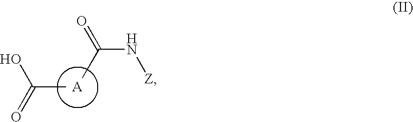

in which A and Z are as defined above
are initially reacted in an inert solvent, with activation of the carboxylic acid function, with a compound of the formula (III)

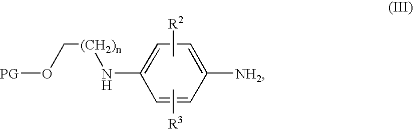

in which n, R² and R³ are as defined above
and
PG represents a hydroxyl protective group, preferably trimethylsilyl or tert-butyl-dimethylsilyl,
   to give compounds of the formula (IV)

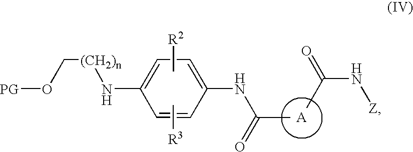

in which n, A, PG, Z, R² and R³ are as defined above,
then either
[A] converted by removal of the protective group PG under customary conditions into compounds of the formula (V)

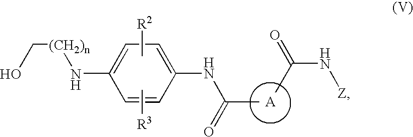

in which n, A, Z, R² and R³ are as defined above,
and the compounds of the formula (V) are then, in an inert solvent in the presence of an acid, converted with cyanogen bromide into compounds of the formula (I-A)

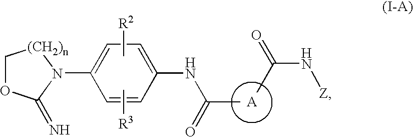

in which n, A, Z, R² and R³ are as defined above,
or
[B] initially reacted in an inert solvent with cyanogen bromide, preferably in the presence of a base, to give compounds of the formula (VI)

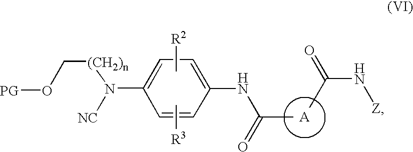

in which n, A, PG, Z, R² and R³ are as defined above,
then converted by removal of the protective group PG into compounds of the formula (VII)

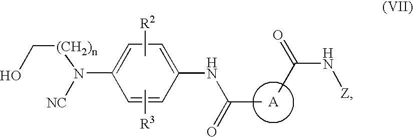

in which n, A, Z, R² and R³ are as defined above,
and the compounds of the formula (VII) are then cyclized in an inert solvent in the presence of an acid to compounds of the formula (I-A)
   and the compounds of the formula (I-A) are, if appropriate, converted with the appropriate (i) solvents and/or (ii) bases or acids into their solvates, salts and/or solvates of the salts.

The compounds of the formula (I) according to the invention in which R¹ does not represent hydrogen can be prepared from the compounds of the formula (V) analogously to processes known from the literature [cf., for example, for R¹=alkanoyl: D. Douglass, J. Amer. Chem. Soc. 1934, 56, 719 and T. Shibanuma, M. Shiono, T. Mukaiyama, Chem. Lett. 1977, 575-576; for R¹=cyano: a) R. Evers, M. Michalik, J. Prakt. Chem. 1991, 333, 699-710; N. Maezaki, A. Furusawa, S. Uchida, T. Tanaka, Tetrahedron 2001, 57, 9309-9316; G. Berecz, J. Reiter, G. Argay, A. Kalman, J. Heterocycl. Chem. 2002, 39, 319-326; b) R. Mohr, A. Buschauer, W. Schunack, Arch. Pharm. (Weinheim Ger.) 1988, 321, 221-227; for R¹=alkyl: a) V. A. Vaillancourt et al., J. Med. Chem. 2001, 44, 1231-1248; b) F. B. Dains et al., J. Amer. Chem. Soc. 1925, 47, 1981-1989; J. Amer. Chem. Soc. 1922, 44, 2637-2643 and T. Shibanuma, M. Shiono, T. Mukaiyama, Chem. Lett. 1977, 575-576.

Inert solvents for the process step (II)+(III)→(IV) are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as ethyl acetate, pyridine, dimethyl sulphoxide, dimethylformamide, N,N′-dimethylpropyleneurea (DMPU), N-methylpyrrolidone (NMP), acetonitrile or acetone. It is also possible to use mixtures of the solvents mentioned. Preference is given to dichloromethane, tetrahydrofuran, dimethylformamide or mixtures of these solvents.

Suitable condensing agents for the amide formation in process step (II)+(III)→(IV) are, for example, carbodiimides, such as N,N′-diethyl-, N,N′-dipropyl-, N,N-diisopropyl-, N,N-dicyclohexylcarbodiimide (DCC), N-(3-dimethylaminoisopropyl)-N′-ethylcarbodiimide hydrochloride (EDC), or phosgene derivatives, such as N,N′-carbonyldiimidazole, or 1,2-oxazolium compounds, such as 2-ethyl-5-phenyl-1,2-oxazolium 3-sulphate or 2-tert-butyl-5-methylisoxazolium perchlorate, or acylamino compounds, such as 2-ethoxy-1-ethoxycarbonyl-1,2-dihydroquinoline, or isobutyl chloroformate, propanephosphonic anhydride, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries, such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or N,N-diisopropylethylamine. Preference is given to using TBTU in combination with N,N-diisopropylethylamine.

The process step (II)+(III)→(IV) is generally carried out in a temperature range of from −20° C. to +60° C., preferably from 0° C. to +40° C. The reaction can be carried out at atmospheric, elevated or reduced pressure (for example from 0.5 to 5 bar). In general, the reaction is carried out at atmospheric pressure.

In process steps (IV)→(V) and (VI)→(VII), the removal of the preferred hydroxyl protective groups (PG) trimethylsilyl or tert-butyldimethylsilyl can preferably be carried out using N-tetrabutylammonium fluoride (TBAF) or, in the case of the reaction (IV)→(V), also using hydrogen fluoride. The reactions are generally carried out in the solvent tetrahydrofuran in a temperature range of from 0° C. to +40° C.

The reaction sequence (VI)→(VII)→(I-A) is particularly preferably carried out using an acid-labile hydroxyl protective group, such as, for example, trimethylsilyl or tert-butyldimethylsilyl, in the presence of an excess of an acid, as a one-pot reaction, without isolation of the intermediate (VII).

Suitable inert solvents for the process steps (V)→(I-A), (IV)→(VI) and (VII)→(I-A) are in particular tetrahydrofuran, dichloromethane or acetonitrile or mixtures of these solvents.

These process steps are generally carried out in a temperature range of from −20° C. to +50° C., preferably from 0° C. to +40° C. The reactions can be carried out under atmospheric, elevated or reduced pressure (for example from 0.5 to 5 bar). In general, they are carried out under atmospheric pressure.

Suitable acids for process steps (V)→(I-A) and (VII)→(I-A) and the reaction sequence (VI)→(VII)→(I-A) are in particular strong inorganic or organic acids, such as, for example, hydrogen fluoride, hydrogen chloride, hydrogen bromide, methanesulphonic acid, trifluoromethanesulphonic acid or trifluoroacetic acid.

The process step (IV)→(VI) is preferably carried out in the presence of a base. Suitable bases are in particular inorganic bases, such as, for example, alkali metal or alkaline earth metal carbonates or bicarbonates, such as lithium carbonate, sodium carbonate, potassium carbonate, calcium carbonate or caesium carbonate or sodium bicarbonate or potassium bicarbonate, or alkali metal hydrides, such as sodium hydride.

The compounds of the formula (II) can be obtained, for example, according to a method from the literature by reacting a carboxylic anhydride of the formula (VIII)

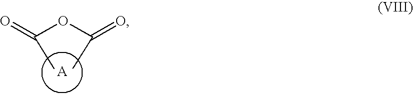

in which A is as defined above
with an amine of the formula (IX)

H₂N-Z (IX),

in which Z is as defined above.

The compounds of the formula (III) can be obtained analogously to methods known from the literature for example by reacting compounds of the formula (X)

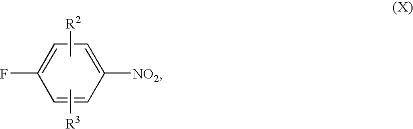

in which R² and R³ are as defined above
with compounds of the formula (XI)

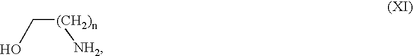

in which n is as defined above
to give compounds of the formula (XII)

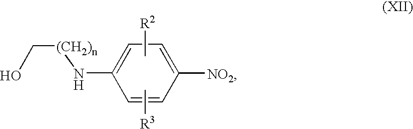

in which n, R² and R³ are as defined above,
subsequent introduction of the hydroxyl protective group PG and then reduction of the nitro group to the amine.

The compounds of the formulae (VIII), (IX), (X) and (XI) are commercially available, known from the literature or can be prepared analogously to methods known from the literature.

The preparation of the compounds according to the invention can be illustrated by the synthesis scheme below:

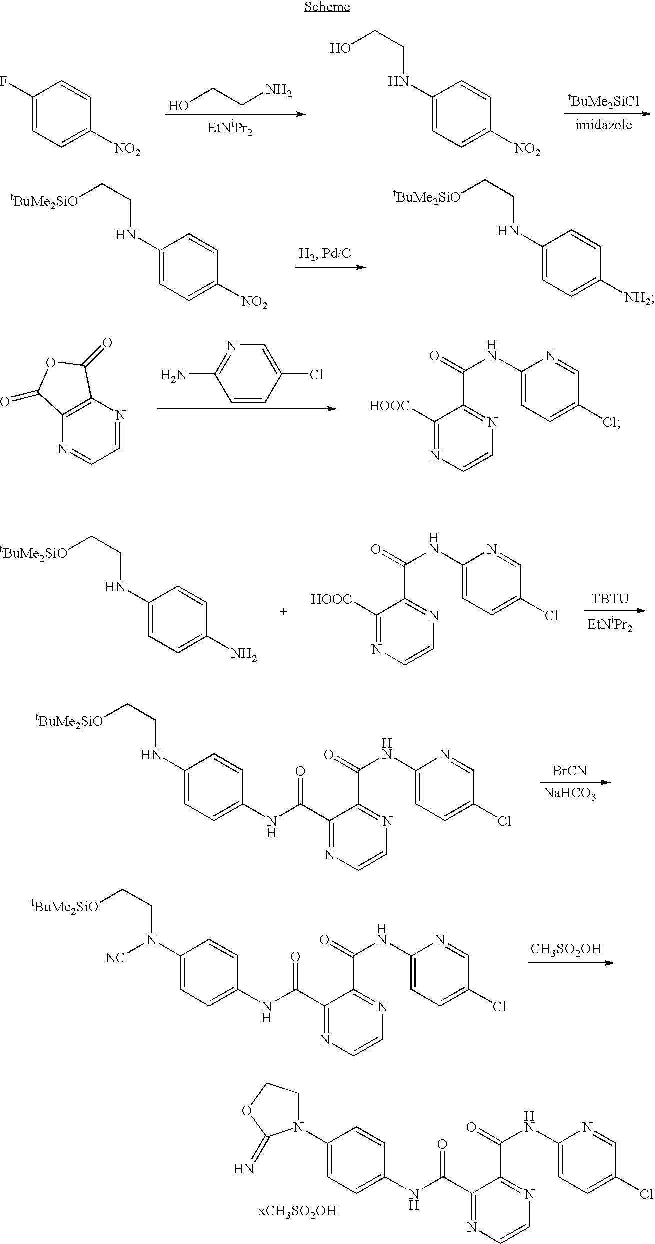

[Abbreviations: ^{t}Bu=tert-butyl; Et=ethyl; Me=methyl; ⁱPr=isopropyl; TBTU=O-(benzotriazol-1-yl)-N,N,N,N′-tetramethyluronium tetrafluoroborate].

The compounds according to the invention have an unforeseeable useful pharmacological activity spectrum.

Accordingly, they are suitable for use as medicaments for the treatment and/or prophylaxis of diseases in humans and animals.

The compounds according to the invention are selective inhibitors of blood coagulation factor Xa which act in particular as anticoagulants.

In addition, the compounds according to the invention have favourable physicochemical properties, such as, for example, good solubility in water and physiological media, which is advantageous for their therapeutic application.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation myocardial infarction (STEMI) or non-ST-elevation myocardial infarction (non-STEMI), stable angina pectoris, unstable angina pectoris, reocclusions and restenoses after coronary interventions such as angioplasty or aortocoronary bypass, peripheral arterial occlusive diseases, pulmonary embolisms, deep vein thromboses and kidney vein thromboses, transitory ischaemic attacks and also thrombotic and thromboembolic stroke.

Accordingly, the substances are also suitable for preventing and treating cardiogenic thromboembolisms, such as, for example, brain ischaemias, stroke and systemic thromboembolisms and ischaemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic haemolytic anaemias, extracorporeal blood circulation, such as haemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumour patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing blood coagulation in vitro, in particular in banked blood or biological samples comprising factor Xa, which method is characterized in that an anticoagulatory effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists);
and also antiarrhythmics.

The present invention furthermore provides medicaments comprising at least one compound according to the invention, usually together with one or more inert non-toxic pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Preference is given to oral or parenteral administration, in particular oral administration.

The compounds according to the invention can be converted into the stated administration forms. This can take place in a manner known per se by mixing with inert, non-toxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavour- and/or odour-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

Abbreviations and acronyms:

| | |
|---|---|
| d | day(s) |
| DCI | direct chemical ionization (in MS) |
| DMF | N,N-dimethylformamide |
| DMSO | dimethyl sulphoxide |
| eq. | equivalent(s) |
| ESI | electrospray ionization (in MS) |
| GC-MS | gas chromatography-coupled mass spectroscopy |
| h | hour(s) |
| HPLC | high pressure, high performance liquid chromatography |
| LC-MS | liquid chromatography-coupled mass spectroscopy |
| min | minute(s) |
| MS | mass spectroscopy |
| NMR | nuclear magnetic resonance spectroscopy |
| RP | reverse phase (in HPLC) |
| RT | room temperature |
| Rₜ | retention time (in HPLC) |
| THF | tetrahydrofuran |

### LC-MS, HPLC and GC-MS Methods:

### Method 1:

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 2:

MS instrument type: Micromass ZQ; HPLC instrument type: HP 1100 Series; UV DAD; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 3:

Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 mil/min, 2.5 min/3.0 min/4.5 min 2 ml/min; oven: 50° C.; UV detection: 208-400 nm.

### Method 4:

Instrument: Micromass Platform LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 5:

Instrument: Micromass Platform LCZ with HPLC Agilent Series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 6:

MS instrument type: Micromass ZQ; HPLC instrument type: Waters Alliance 2795; column: Merck Chromolith SpeedROD RP-18e 50 mm×4.6 mm; mobile phase A: 1 l of water+0.5 ml of 50% strength formic acid, mobile phase B: 1 l of acetonitrile+0.5 ml of 50% strength formic acid; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

### Method 7:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

### Method 8:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.

### Method 9:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### Method 10:

Instrument: Micromass GCT, GC6890; column: Restek RTX-35MS, 30 m×250 μm×0.25 μm; constant flow rate with helium: 0.88 ml/min; oven: 60° C.; inlet: 250° C.; gradient: 60° C. (maintained for 0.30 min), 50° C./min ˜120° C., 16° C./min ˜250° C., 30° C./min→300° C. (maintained for 1.7 min).

### Starting Materials and Intermediates:

### Example 1A

### N-(2-{[tert-Butyl(dimethyl)silyl]oxy}ethyl)benzene-1,4-diamine

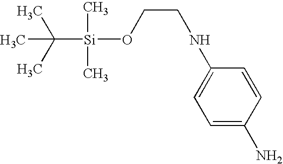

### Step a): 2-[(4-Nitrophenyl)amino]ethanol

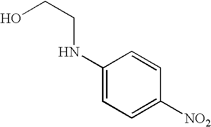

130 ml (2.15 mol, 3 eq.) of 2-aminoethanol and 274 ml (1.57 mol, 2.2 eq.) of N,N-di-isopropylethylamine are added to a solution of 101 g (716 mmol) of 4-fluoronitrophenol in 500 ml of ethanol. The reaction mixture is stirred at 50° C. overnight, a further 86 ml (1.43 mol, 2.0 eq.) of 2-aminoethanol and 249 ml (1.43 mol, 2.0 eq.) of N,N-diisopropylethylamine are then added and the mixture is stirred at 50° C. for a further 12 h. The reaction solution is concentrated under reduced pressure and the residue is triturated with 600 ml of water. The precipitate formed is filtered off, washed repeatedly with water and dried.

Yield: 127 g (97% of theory).

LC-MS (method 5): Rₜ=2.32 min;

MS (ESIpos): m/z=183 [M+H]⁺;

¹H NMR (300 MHz, DMSO-d₆): δ=7.99 (d, 2H), 7.30 (t, 1H), 6.68 (d, 2H), 4.82 (t, 1H), 3.63-3.52 (m, 2H), 3.30-3.19 (m, 2H).

### Step b): N-(2-{[tert-Butyl(dimethyl)silyl]oxy}ethyl)-4-nitroaniline

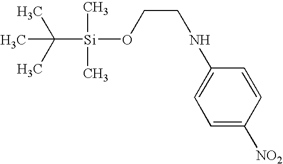

At RT, 30.6 g (203 mmol, 1.2 eq.) of tert-butyldimethylchlorosilane and 17.3 g (254 mmol, 1.5 eq.) of imidazole are added to a solution of 30.8 g (169 mmol) of 2-[(4-nitrophenyl)amino]ethanol in 300 ml of DMF, and the mixture is stirred at RT for 2.5 h. The reaction mixture is concentrated under reduced pressure and the residue is dissolved in 200 ml of dichloromethane and 100 ml of water. After phase separation, the aqueous phase is extracted three times with in each case 80 ml of dichloromethane. The combined organic phases are washed with 100 ml of saturated aqueous sodium chloride solution, dried over sodium sulphate, filtered and concentrated under reduced pressure.

Yield: 49.7 g (quant.)

LC-MS (method 3): Rₜ=3.09 min;

MS (ESIpos): m/z=297 [M+H]⁺;

¹H NMR (300 MHz, DMSO-d₆): δ=7.98 (d, 2H), 7.29 (t, 1H), 6.68 (d, 2H), 3.77-3.66 (m, 2H), 3.35-3.24 (m, 2H), 0.81 (s, 9H), 0.0 (s, 6H).

### Step c): N-(2-{[tert-Butyl(dimethyl)silyl]oxy}ethyl)benzene-1,4-diamine

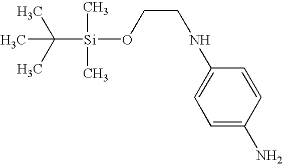

Under argon, 4 g of palladium-on-carbon (10%) are added to a solution of 59.5 g (201 mmol) of N-(2-{[tert-butyl(dimethyl)silyl]oxy}ethyl)-4-nitroaniline in 500 ml of ethanol, and the mixture is hydrogenated under an atmosphere of hydrogen at RT and atmospheric pressure. The catalyst is removed through a filter layer and washed with ethanol, and the filtrate is concentrated under reduced pressure.

Yield: 53 g (quant.)

LC-MS (method 2): Rₜ=1.83 min;

MS (ESIpos): m/z=267 [M+H]⁺;

¹H NMR (300 MHz, DMSO-d₆): δ=6.42-6.30 (m, 4H), 4.48 (t, 1H), 4.21 (br. s, 2H), 3.68-3.58 (m, 2H), 3.04-2.93 (m, 2H), 0.82 (s, 9H), 0.0 (s, 6H).

### Example 2A

### N-(3-{[tert-Butyl(dimethyl)silyl]oxy}propyl)benzene-1,4-diamine

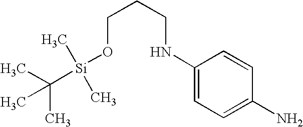

The title compound is prepared by a reaction sequence analogous to the one described in Example 1A.

LC-MS (method 6): Rₜ=1.73 min;

MS (ESIpos): m/z=281 [M+H]⁺;

¹H NMR (400 MHz, DMSO-d₆): δ=6.39 (d, 2H), 6.30 (d, 2H), 4.56 (br. s, 1H), 4.19 (br. s, 2H), 3.69-3.60 (m, 2H), 2.97-2.88 (m, 2H), 1.70-1.60 (m, 2H), 0.83 (s, 9H), 0.0 (s, 6H).

### Example 3A

### 3-{[(5-Chloropyridin-2-yl)amino]carbonyl}pyrazine-2-carboxylic acid

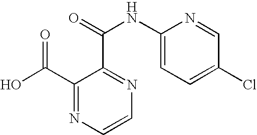

68.0 g (0.53 mol) of 2-amino-5-chloropyridine are dissolved in 1100 ml of THF, and 95.3 g (0.63 mol) of 2,3-pyrazinedicarboxylic anhydride are added a little at a time. The suspension is stirred at room temperature for one hour. The precipitate is then filtered off. The filtrate is concentrated and the residue is combined with the precipitate. The product is triturated with diethyl ether, filtered again and dried under reduced pressure.

Yield: 154 g (99% of theory)

HPLC (method 9): Rₜ=3.50 min;

MS (ESIpos): m/z=279 [M+H]⁺;

¹H NMR (300 MHz, DMSO-d₆): δ=13.89 (br. s, 1H), 11.07 (s, 1H), 8.90 (dd, 2H), 8.44 (s, 1H), 8.20 (d, 1H), 8.01 (dd, 1H).

### Example 4A

### 3-{[(5-Methylpyridin-2-yl)amino]carbonyl}pyrazine-2-carboxylic acid

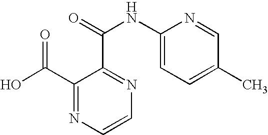

2.5 g (23.3 mmol) of 5-methylpyridine-2-amine and 3.5 g (23.3 mmol) of 2,3-pyrazinedicarboxylic anhydride are reacted analogously to the method described for Example 3A.

Yield: 5.2 g (94% pure, 82% of theory)

LC-MS (method 5): Rₜ=1.96 min;

MS (ESIpos): m/z=215 [M+H—CO₂]⁺;

¹H NMR (300 MHz, DMSO-d₆): δ=10.71 (s, 1H), 8.89 (d, 2H), 8.22 (s, 1H), 8.08 (d, 1H), 7.70 (s, 1H), 2.30 (s, 3H).

### Example 5A

### 3-{[(5-Cyanopyridin-2-yl)amino]carbonyl}pyrazine-2-carboxylic acid

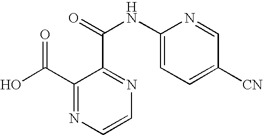

1.0 g (8.4 mmol) of 6-aminonicotinonitrile and 1.3 g (8.4 mmol) of 2,3-pyrazinedicarboxylic anhydride are reacted analogously to the method described for Example 3A. The crude product is purified by flash chromatography on silica gel (mobile phase: dichloromethane/methanol 120:1→40:1).

Yield: 765 mg (90% pure, 30% of theory)

HPLC (method 7): Rₜ=3.21 min;

MS (DCI, NH₃): m/z=287 [M+NH₄]⁺;

¹H NMR (300 MHz, DMSO-d₆): δ=14.1 (br. s, 1H), 11.44 (s, 1H), 8.90 (dd, 2H), 8.85 (s, 1H), 8.40-8.22 (m, 2H).

### Example 6A

### 3-{[(4-Cyanophenyl)amino]carbonyl}pyrazine-2-carboxylic acid

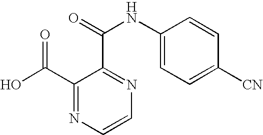

1.0 g (8.5 mmol) of 4-aminobenzonitrile and 1.3 g (8.5 mmol) of 2,3-pyrazinedicarboxylic anhydride are reacted analogously to the method described for Example 3A.

Yield: 2.1 g (92% of theory)

LC-MS (method 3): Rₜ=1.06 min;

MS (ESIpos): m/z=225 [M+H—CO₂]⁺;

¹H NMR (300 MHz, DMSO-d₆): δ=13.92 (br. s, 1H), 11.22 (s, 1H), 8.92 (dd, 2H), 7.99 (d, 2H), 7.87 (d, 2H).

### Example 7A

### 3-{[(4-Ethynylphenyl)amino]carbonyl}pyrazine-2-carboxylic acid

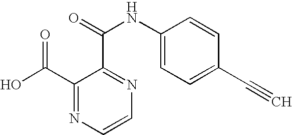

500 mg (4.27 mmol) of 4-ethynylaniline and 641 mg (4.27 mmol) of 2,3-pyrazine-dicarboxylic anhydride are reacted analogously to the method described for Example 3A.

Yield: 1.08 g (96% pure, 91% of theory)

LC-MS (method 3): Rₜ=1.49 min;

MS (ESIpos): m/z=224 [M+H—CO₂]⁺.

### Example 8A

### 3-{[(5-Chloropyridin-2-yl)amino]carbonyl}thiophene-2-carboxylic acid and 2-{[(5-chloropyridin-2-yl)amino]carbonyl}thiophene-3-carboxylic acid (mixture of regioisomers)

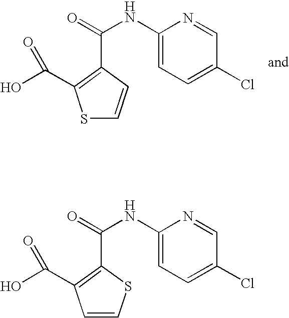

A solution of 1.17 g (9.08 mmol) of 2-amino-5-chloropyridine and 1.40 g (9.08 mmol) of thieno[2,3-c]furan-4,6-dione [Reinecke, M. G.; Newsom, J. G.; Chen, L.-J., J. Am. Chem. Soc. 1981, 103, 2760-2769] in 30 ml of THF is stirred at RT for 20 h. The precipitate formed is filtered off, washed with THF and dried under reduced pressure.

Yield: 1.05 g (41% of theory)

LC-MS (method 3): Rₜ=1.77 min and 2.03 min;

MS (ESIpos): m/z=283 [M+H]⁺.

### Example 9A

### 4-{[(5-Chloropyridin-2-yl)amino]carbonyl}-2-methyl-1,3-thiazole-5-carboxylic acid and 5-{[(5-chloropyridin-2-yl)amino]carbonyl}-2-methyl-1,3-thiazole-4-carboxylic acid (mixture of regioisomers)

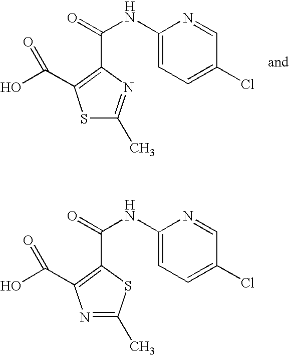

### Step a): 2-Methylfuro[3,4-d][1,3]thiazole-4,6-dione

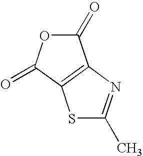

A solution of 5.0 g (26.9 mmol) of 2-methyl-1,3-thiazole-4,5-dicarboxylic acid [Rublew et al., Justus Liebigs Ann. Chem. 1890, 259, 272-274] in 20 ml of thionyl chloride is stirred under reflux for 2 h and then concentrated under reduced pressure and dried. The crude product (5.45 g) is used without further purification for the next step.

GC-MS (method 10): Rₜ=7.57 min;

MS (ESIpos): m/z=169 [M]⁺.

### Step b): 4-{[(5-Chloropyridin-2-yl)amino]carbonyl}-2-methyl-1,3-thiazole-5-carboxylic acid and 5-{[(5-chloropyridin-2-yl)amino]carbonyl}-2-methyl-1,3-thiazole-5-carboxylic acid (mixture of regioisomers)

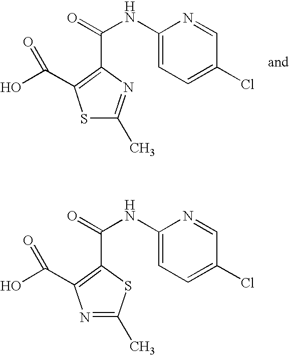

4.7 ml (26.8 mmol) of N,N-diisopropylethylamine are added to a solution of 4.54 g (26.8 mmol) of 2-methylfuro[3,4-d][1,3]thiazole-4,6-dione and 3.45 g (26.8 mmol) of 2-amino-5-chloropyridine in 161 ml of THF, and the mixture is stirred at RT for 17 h. The reaction solution is concentrated under reduced pressure and the crude product (12.3 g) is used as a mixture of regioisomers without further purification for the next reaction.

LC-MS (method 3): Rₜ=1.99 min;

MS (ESIpos): m/z=253 [M+H—CO₂]⁺.

### Example 10A

### Phenyl 5,10-dioxo-5H,10H-diimidazo[1,5-a:1′,5′-d]pyrazine-1,6-dicarboxylate

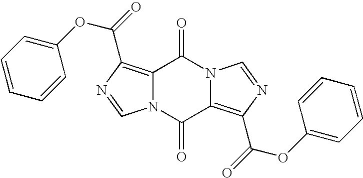

### Step a): 5,10-Dioxo-5H,10H-diimidazo[1,5-a:1′,5′-d]pyrazine-1,6-dicarbonyl chloride

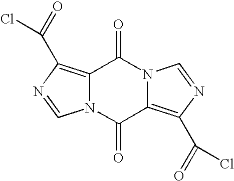

A solution of 1.0 g (6.5 mmol) of 4,5-imidazoledicarboxylic acid in 5 ml of toluene is initially charged in a flask dried by heating, 2.8 ml of thionyl chloride and 30 μl of dimethylformamide are added and the reaction mixture is stirred under reflux for 16 h. After cooling, the precipitate formed is filtered off, washed twice with toluene and dried under reduced pressure. The crude product is used without further purification for the next step.

### Step b): Phenyl 5,10-dioxo-5H,10H-diimidazo[1,5-a:1′,5′-d]pyrazine-1,6-dicarboxylate

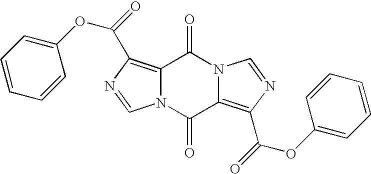

Under argon, 568 mg (6.03 mmol) of phenol, at RT, and then, at 0° C. over a period of 2 min, 490 μl (6.03 mmol) of pyridine are added to a solution of 899 mg (2.87 mmol) of 5,10-dioxo-5H,10H-diimidazo[1,5-a:1′,5′-d]pyrazine-1,6-dicarbonyl chloride in 17 ml of dichloromethane. The reaction mixture is stirred at RT for 2 h. The precipitate formed is filtered off, washed twice with dichloromethane and dried under reduced pressure.

Yield: 1.08 g (87% of theory)

¹H NMR (300 MHz, DMSO-d₆): δ=9.14 (s, 2H), 7.59-7.48 (m, 4H), 7.41-7.31 (m, 6H).

### Example 11A

Methyl 5,10-dioxo-5H,10H-diimidazo[1,5-a:1′,5′-d]pyrazine-1,6-dicarboxylate

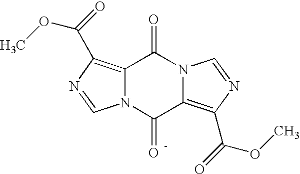

Under argon, 70 μl (1.68 mmol) of methanol, at RT, and then, at 0° C. over a period of 2 min, 140 μl (1.68 mmol) of pyridine are added to a solution of 250 mg (0.80 mmol) of 5,10-dioxo-5H,10H-diimidazo[1,5-a:1′,5′-d]pyrazine-1,6-dicarbonyl chloride in 5 ml of dichloromethane. The reaction mixture is stirred at RT for 1 h. The precipitate formed is filtered off, washed twice with dichloromethane and dried under reduced pressure.

Yield: 212 mg (87% of theory)

LC-MS (method 3): Rₜ=1.06 min;

MS (ESIpos): m/z=305 [M+H]⁺;

¹H NMR (400 MHz, DMSO-d₆): δ=8.98 (s, 2H), 3.92 (s, 6H).

### Example 12A

### 4-{[(4-Chlorophenyl)amino]carbonyl}-1-methyl-1H-pyrrole-3-carboxylic acid

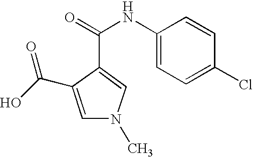

### Step a): Ethyl 1-methyl-1H-pyrrole-3,4-dicarboxylate

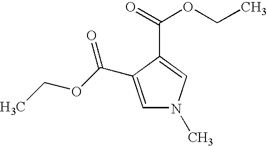

At RT, 852 mg (21.3 mmol) of sodium hydride (60% in mineral oil), a little at a time, and then 1.33 ml (21.3 mmol) of iodomethane, dropwise, are added to a solution of 1.5 g (7.1 mmol) of ethyl 3,4-pyrroledicarboxylate in 5 ml of dimethylformamide. The reaction mixture is stirred at RT overnight, and 0.5 N hydrochloric acid and dichloromethane are then added. After phase separation, the aqueous phase is extracted with dichloromethane and the combined organic phases are dried over sodium sulphate, filtered and concentrated under reduced pressure. The title compound is isolated by flash chromatography on silica gel (mobile phase: cyclohexane/ethyl acetate 2:1).

Yield: 666 mg (98% pure, 41% of theory)

LC-MS (method 3): Rₜ=1.78 min;

MS (ESIpos): m/z=226 [M+H]⁺.

### Step b): 1-Methyl-1H-pyrrole-3,4-dicarboxylic acid

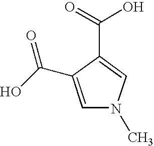

At RT, 123 mg (5.13 mmol) of lithium hydroxide are added to a solution of 578 mg (2.57 mmol) of ethyl 1-methyl-1H-pyrrole-3,4-dicarboxylate in 20 ml of THF/water (3:1), and the mixture is stirred at 60° C. overnight. The THF is removed under reduced pressure and the residue is acidified to pH 1 using 1 N hydrochloric acid. The precipitate formed is filtered off and dried under reduced pressure.

Yield: 346 mg (80% of theory)

LC-MS (method 5): Rₜ=1.99 min;

MS (ESIpos): m/z=170 [M+H]⁺.

### Step c): 5-Methyl-1H-furo[3,4-c]pyrrole-1,3(5H)dione

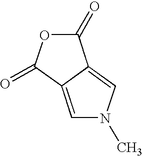

At RT, a solution of 441 mg (2.14 mmol) of N,N′-dicyclohexylcarbodiimide in 2.5 ml of THF is added to a solution of 329 mg (1.95 mmol) of 1-methyl-1H-pyrrole-3,4-dicarboxylic acid in 5.5 ml of THF. The reaction mixture is stirred under reflux overnight. After cooling, the precipitate formed is filtered off. The mother liquor is concentrated under reduced pressure and the crude product is used without further purification for the next step.

Yield: 360 mg (78% pure, 94% of theory)

GC-MS (method 10): Rₜ=10.37 min;

MS (ESIpos): m/z=151 [M]⁺.

### Step d): 4-{[(4-Chlorophenyl)amino]carbonyl}-1-methyl-1H-pyrrole-3-carboxylic acid

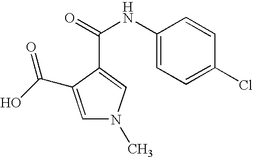

At RT, 169 mg (1.32 mmol) of 4-chloroaniline are added to a solution of 200 mg (1.32 mmol) of 5-methyl-1H-furo[3,4-c]pyrrole-1,3(5H)-dione in 5 ml of THF, and the mixture is stirred at RT overnight. The precipitate formed is filtered off and dried.

Yield: 205 mg (56% of theory)

LC-MS (method 2): Rₜ=2.21 min;

MS (ESIpos): m/z=279 [M+H]⁺.

### Working Examples

### General Method 1

### Amide Coupling

The carboxylic acid in question and N,N-diisopropylethylamine (1.05 eq.) are initially charged in dichloromethane and stirred at RT for 15 min. A solution of the aniline derivative (1.0 eq.) in dichloromethane is then added dropwise. O-(benzotriazol-1-yl)-N,N,N′,N′-tetramethyluronium tetrafluoroborate (1.05 eq.) is added, and the mixture is stirred at room temperature overnight. The reaction solution is then washed with water, with saturated aqueous sodium bicarbonate solution and again with water. The solvent is removed under reduced pressure, and ethyl acetate is added to the residue. The precipitated solid is filtered off and washed with pentane. The filtrate is concentrated under reduced pressure and the residue is purified by column chromatography.

### Example 1

### N-(5-Chloropyridin-2-yl)-N′-[4-(2-imino-1,3-oxazolidin-3-yl)phenyl]pyrazine-2,3-dicarboxamide methanesulphonate

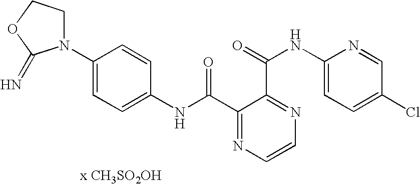

### Step a): N-{4-[(2-{[tert-Butyl(dimethyl)silyl]oxy}ethyl)amino]phenyl}-N′-(5-chloropyridin-2-yl)pyrazine-2,3-dicarboxamide

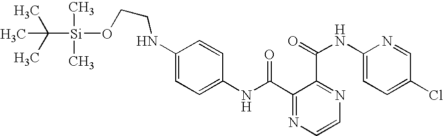

According to the General Method 1, 104.5 g (0.38 mol) of the compound from Example 3A are reacted with 100.0 g (0.38 mol) of the compound from Example 1A.

Yield: 101.3 g (51% of theory)

LC-MS (method 3): Rₜ=2.96 min;

MS (ESIpos): m/z=527 [M+H]⁺;

¹H NMR (400 MHz, DMSO-d₆): δ=11.07 (s, 1H), 10.37 (s, 1H), 8.85 (s, 2H), 8.38 (s, 1H), 8.21 (d, 1H), 7.95 (d, 1H), 7.45 (d, 2H), 6.53 (d, 2H), 5.40 (t, NH), 3.67 (t, 2H), 3.10 (dt, 2H), 0.83 (s, 9H), 0.00 (s, 6H).

### Step b): N-{4-[(2-{[tert-Butyl(dimethyl)silyl]oxy}ethyl)(cyano)amino]phenyl}-N′-(5-chloropyridin-2-yl)pyrazine-2,3-dicarboxamide

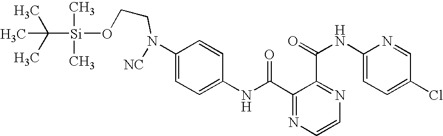

65 ml of THF are added to 15.0 g (28.5 mmol) of N-{4-[(2-{[tert-butyl(dimethyl)silyl]oxy}ethyl)amino]phenyl}-N′-(5-chloropyridin-2-yl)pyrazine-2,3-dicarboxamide, and 7.17 g (85.4 mmol) of sodium bicarbonate are added. 3.6 g (34.2 mmol) of cyanogen bromide are then added. The suspension is stirred at 40° C. for 12 h. 250 ml of water and 300 ml of dichloromethane are added. The organic phase is separated off, washed with 300 ml of a saturated aqueous sodium bicarbonate solution, dried over magnesium sulphate, filtered and concentrated under reduced pressure. The residue is triturated with diethyl ether, filtered off and dried under reduced pressure.

Yield: 13.7 g (82% of theory, 94% pure)

HPLC (method 8): Rₜ=5.72 min;

MS (ESIpos): m/z=552 [M+H]⁺;

¹H NMR (300 MHz, DMSO-d₆): δ=11.16 (s, 1H), 10.86 (s, 1H), 9.05 (s, 2H), 8.44 (s, 1H), 8.27 (d, 1H), 8.03 (dd, 1H), 7.86 (d, 2H), 7.23 (d, 2H), 3.80-3.90 (m, 4H), 0.86 (s, 9H), 0.00 (s, 6H).

### Step c): N-(5-Chloropyridin-2-yl)-N′-[4-(2-imino-1,3-oxazolidin-3-yl)phenyl]-pyrazine-2,3-dicarboxamide methanesulphonate

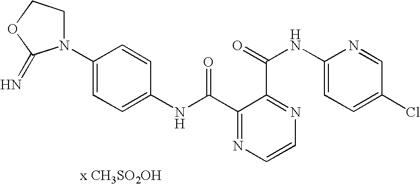

170 ml of acetonitrile and 11.5 g (120.0 mmol) of methanesulphonic acid are added to 32.0 g (58 mmol) of N-{4-[(2-{[tert-butyl(dimethyl)silyl]oxy}ethyl)(cyano)amino]phenyl}-N′-(5-chloropyridin-2-yl)pyrazine-2,3-dicarboxamide, and the mixture is stirred at RT for 35 h. The solid is filtered off and washed three times with acetonitrile. The solid is then dried under reduced pressure.

Yield: 25.1 g (81% of theory)

HPLC (method 7): Rₜ=3.65 min;

MS (ESIpos): m/z=438 [M+H]⁺ (free base);

¹H NMR (300 MHz, DMSO-d₆): δ=11.13 (s, 1H), 11.07 (s, 1H), 9.58 (s, 1H), 8.97 (s, 2H), 8.78-8.85 (m, 1H), 8.43 (s, 1H), 8.20 (d, 1H), 8.01 (d, 1H), 7.96 (d, 2H), 7.51 (d, 2H), 4.85 (t, 2H), 4.23 (t, 2H), 2.29 (s, 3H).

The following salts are prepared analogously by reaction with the appropriate acids:

### Example 2

### N-(5-Chloropyridin-2-yl)-N′-[4-(2-imino-1,3-oxazolidin-3-yl)phenyl]pyrazine-2,3-dicarboxamide hydrobromide

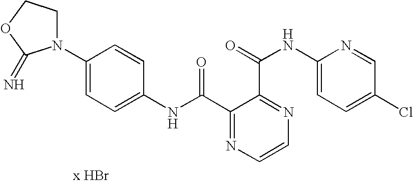

LC-MS (method 2): Rₜ=1.39 min;

MS (ESIpos): m/z=438 [M+H]⁺ (free base);

¹H NMR (300 MHz, DMSO-d₆): δ=11.12 (s, 1H), 10.97 (s, 1H), 8.96 (s, 2H), 8.73-8.69 (m, 1H), 8.43 (s, 1H), 8.22 (d, 1H), 8.00 (dd, 1H), 7.90 (d, 2H), 7.57 (d, 2H), 4.71 (t, 2H), 4.16 (t, 2H).

### Example 3

### N-(5-Chloropyridin-2-yl)-N′-[4-(2-imino-1,3-oxazolidin-3-yl)phenyl]pyrazine-2,3-dicarboxamide hydrochloride

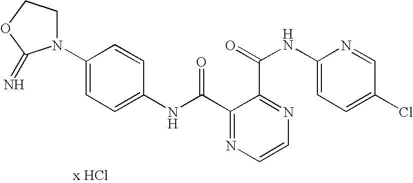

LC-MS (method 2): Rₜ=1.16 min;

MS (ESIpos): m/z=438 [M+H]⁺ (free base);

¹H NMR (300 MHz, DMSO-d₆): δ=11.13 (s, 1H), 11.01 (s, 1H), 9.64-9.60 (m, 1H), 8.97 (s, 2H), 8.85-8.81 (m, 1H), 8.43 (s, 1H), 8.21 (d, 1H), 8.00 (dd, 1H), 7.96 (d, 2H), 7.51 (d, 2H), 4.85 (t, 2H), 4.23 (t, 2H).

### Example 4

### N-(5-Chloropyridin-2-yl)-N′-[4-(2-imino-1,3-oxazolidin-3-yl)phenyl]pyrazine-2,3-dicarboxamide

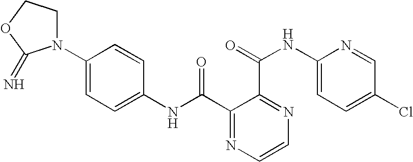

The free base is obtained by stirring a solution of the appropriate salt (Example 1, 2 or 3) in THF with saturated aqueous sodium bicarbonate solution, followed by extraction with dichloromethane.

LC-MS (method 5): Rₜ=2.55 min;

MS (ESIpos): m/z=438 [M+H]⁺;

¹H NMR (400 MHz, DMSO-d₆): δ=11.12 (s, 1H), 10.77 (s, 1H), 8.92 (s, 2H), 8.41 (s, 1H), 8.24 (d, 1H), 8.00 (dd, 1H), 7.79 (d, 2H), 7.70 (d, 2H), 4.47-4.30 (m, 2H), 4.19-3.92 (m, 2H).

### Example 5

### N-(5-Chloropyridin-2-yl)-N′-[4-(2-imino-1,3-oxazinan-3-yl)phenyl]pyrazine-2,3-dicarboxamide methanesulphonate

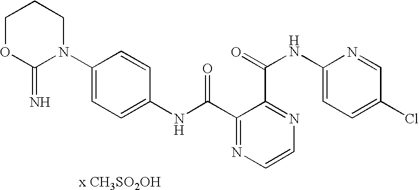

### Step a): N-{4-[(3-{[tert-Butyl(dimethyl)silyl]oxy}propyl)amino]phenyl}-N′-(5-chloropyridin-2-yl)pyrazine-2,3-dicarboxamide

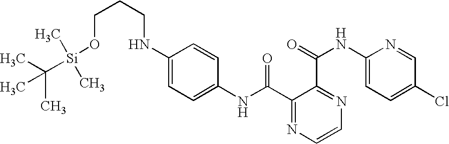

According to the General Method 1, 8.24 g (29.4 mmol) of the compound from Example 2A are reacted with 8.19 g (29.4 mmol) of the compound from Example 3A.

Yield: 10.7 g (80% pure, 54% of theory)

LC-MS (method 3): Rₜ=2.85 min;

MS (ESIpos): m/z=541 [M+H]⁺;

¹H NMR (300 MHz, DMSO-d₆): δ=11.08 (s, 1H), 10.49 (s, 1H), 8.88 (s, 2H), 8.40 (d, 1H), 8.23 (d, 1H), 7.98 (dd, 1H), 7.48 (d, 2H), 6.50 (d, 2H), 5.49 (t, 1H), 3.68 (t, 2H), 3.04 (dt, 2H), 1.71 (q, 2H), 0.88 (s, 9H), 0.02 (s, 6H).

### Step b): N-{4-[(3-{[tert-Butyl(dimethyl)silyl]oxy}propyl)(cyano)amino]phenyl}-N′-(5-chloropyridin-2-yl)pyrazine-2,3-dicarboxamide

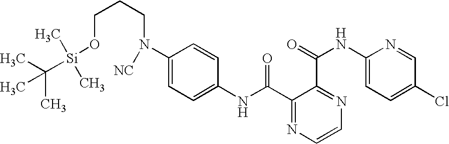

At RT, 2.8 g (33.3 mmol, 3 eq.) of sodium bicarbonate and 4.4 ml (13.3 mmol, 1.2 eq.) of a 3 M solution of cyanogen bromide in dichloromethane are added to a solution of 7.5 g (11.1 mmol) of N-{4-[(3-{[tert-butyl(dimethyl)silyl]oxy}propyl)amino]phenyl}-N′-(5-chloropyridin-2-yl)pyrazine-2,3-dicarboxamide in 40 ml of THF. The reaction mixture is stirred at 40° C. for 8 h, and 155 ml of water and 190 ml of dichloromethane are then added. After phase separation, the organic phase is washed with 190 ml of a saturated aqueous sodium bicarbonate solution, dried over magnesium sulphate, filtered and concentrated under reduced pressure. The residue is triturated with diisopropyl ether, filtered off and dried under reduced pressure.

Yield: 6.3 g (98% pure, 99% of theory)

LC-MS (method 2): Rₜ=3.13 min;

MS (ESIpos): m/z=566 [M+H]⁺;

¹H NMR (300 MHz, DMSO-d₆): δ=11.08 (s, 1H), 10.81 (s, 1H), 8.90 (s, 2H), 8.39 (s, 1H), 8.21 (d, 1H), 7.96 (dd, 1H), 7.80 (d, 2H), 7.14 (d, 2H), 3.73-3.60 (m, 4H), 1.82 (q, 1H), 0.82 (s, 9H), 0.00 (s, 6H).

### Step c): N-(5-Chloropyridin-2-yl)-N′-[4-(2-imino-1,3-oxazinan-3-yl)phenyl]pyrazine-2,3-dicarboxamide methanesulphonate

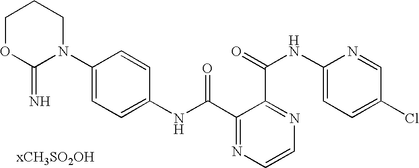

At RT, 555 μl (8.55 mmol, 2.2 eq.) of methanesulphonic acid are added to a solution of 2.20 g (3.89 mmol) of N-{4-[(3-{[tert-butyl(dimethyl)silyl]oxy}propyl)(cyano)-amino]phenyl}-N′-(5-chloropyridin-2-yl)pyrazine-2,3-dicarboxamide in 100 ml of acetonitrile, the mixture is stirred at RT overnight, a further 126 μl (1.94 mmol, 0.5 eq.) of methanesulphonic acid are added and the mixture is again stirred overnight. The reaction mixture is concentrated and the crude product is purified by flash chromatography (silica gel 60, mobile phase: dichloromethane/methanol 20:1→5:1).

Yield: 1.54 g (72% of theory)

HPLC (method 9): Rₜ=3.73 min;

MS (ESIpos): m/z=452 [M+H]⁺;

¹H NMR (300 MHz, DMSO-d₆): δ=11.14 (s, 1H), 11.08 (s, 1H), 8.96 (s, 2H), 8.75 (br. s, 1H), 8.44 (s, 1H), 8.21 (d, 1H), 8.02 (d, 1H), 7.98 (d, 2H), 7.74 (br. s, 1H), 7.51 (d, 2H), 4.60 (dd, 2H), 3.65 (dd, 2H), 2.33 (s, 3H), 2.29 (m, 2H).

### Example 6

### N-[4-(2-Imino-1,3-oxazolidin-3-yl)phenyl]-N′-(5-methylpyridin-2-yl)pyrazine-2,3-dicarboxamide methanesulphonate

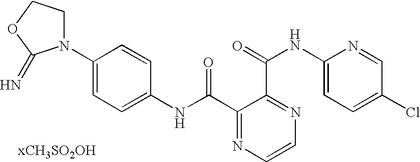

### Step a): N-{4-[(2-{[tert-Butyl(dimethyl)silyl]oxy}ethyl)amino]phenyl}-N′-(5-methylpyridin-2-yl)pyrazine-2,3-dicarboxamide

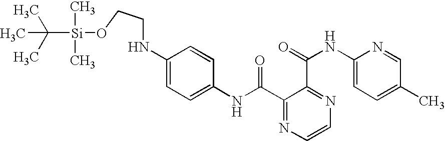

According to the General Method 1, 1.0 g (3.9 mmol) of the compound from Example 4A is reacted with 1.0 g (3.9 mmol) of the compound from Example 1A.

Yield: 737 mg (38% of theory)

LC-MS (method 3): Rₜ=2.49 min;

MS (ESIpos): m/z=507 [M+H]⁺.

### Step b): N-{4-[(2-{[tert-Butyl(dimethyl)silyl]oxy}ethyl)(cyano)amino]phenyl}-N′-(5-methylpyridin-2-yl)pyrazine-2,3-dicarboxamide

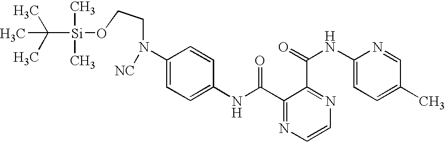

At RT, 159 mg (1.89 mmol) of sodium bicarbonate and 230 μl (0.69 mmol) of a 3 M solution of cyanogen bromide in dichloromethane are added to a solution of 320 mg (0.63 mmol) of N-{4-[(2-{[tert-butyl(dimethyl)silyl]oxy}ethyl)amino]phenyl}-N′-(5-methylpyridin-2-yl)pyrazine-2,3-dicarboxamide in 4 ml of THF. The suspension is stirred at 40° C. for 16 h and then diluted with 3 ml of water and 5 ml of dichloromethane. After phase separation, the organic phase is washed with saturated aqueous sodium bicarbonate solution, dried over magnesium sulphate, filtered and concentrated under reduced pressure. The residue is triturated in diisopropyl ether and filtered, and the solid is dried under reduced pressure.

Yield: 219 mg (65% of theory)

LC-MS (method 2): Rₜ=2.83 min;

MS (ESIpos): m/z=532 [M+H]⁺;

¹H NMR (400 MHz, DMSO-d₆): δ=10.80 (s, 2H), 8.95 (s, 2H), 8.22 (s, 1H), 8.12 (d, 1H), 7.85 (d, 2H), 7.70 (d, 1H), 7.20 (d, 2H), 3.90-3.79 (m, 4H), 2.31 (s, 3H), 0.86 (s, 9H), 0.0 (s, 6H).

### Step c): N-[4-(2-Imino-1,3-oxazolidin-3-yl)phenyl]-N′-(5-methylpyridin-2-yl)pyrazine-2,3-dicarboxamide methanesulphonate

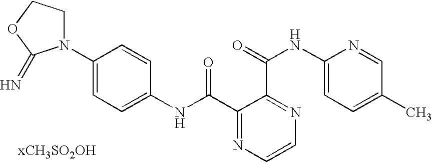

At RT, 13 μl (0.20 mmol) of methanesulphonic acid are added to a solution of 50 mg (0.09 mmol) of N-{4-[(2-{[tert-butyl(dimethyl)silyl]oxy}ethyl)(cyano)amino]phenyl}-N′-(5-methylpyridin-2-yl)pyrazine-2,3-dicarboxamide in 5 ml of acetonitrile, and the mixture is stirred at RT overnight. The reaction mixture is then concentrated under reduced pressure and the residue is dried.

Yield: 47 mg (quant.)

LC-MS (method 2): Rₜ=1.29 min;

MS (ESIpos): m/z=418 [M+H]⁺ (free base);

¹H NMR (400 MHz, DMSO-d₆): δ=11.02 (s, 1H), 10.89 (s, 1H), 9.58 (s, 1H), 8.96 (s, 2H), 8.80 (s, 1H), 8.22 (s, 1H), 8.06 (d, 1H), 7.94 (d, 2H), 7.73 (d, 1H), 7.51 (d, 2H), 4.85 (t, 2H), 4.23 (t, 2H), 2.33 (s, 3H), 2.29 (s, 3H).

### Example 7

### N-(5-Cyanopyridin-2-yl)-N′-[4-(2-imino-1,3-oxazolidin-3-yl)phenyl]pyrazine-2,3-dicarboxamide methanesulphonate

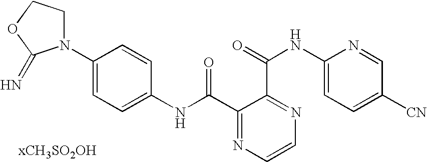

### Step a): N-{4-[(2-{[tert-Butyl(dimethyl)silyl]oxy}ethyl)amino]phenyl}-N′-(5-cyanopyridin-2-yl)pyrazine-2,3-dicarboxamide

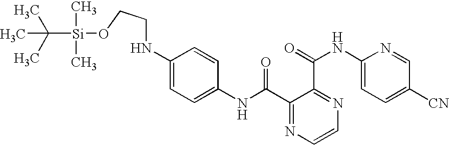

According to the General Method 1, 250 mg (0.93 mmol) of the compound from Example 5A are reacted with 247 mg (0.93 mmol) of the compound from Example 1A.

Yield: 205 mg (97% pure, 41% of theory)

LC-MS (method 1): Rₜ=2.63 min;

MS (ESIpos): m/z=518 [M+H]⁺.

### Step b): N-{4-[(2-{[tert-Butyl(dimethyl)silyl]oxy}ethyl)(cyano)amino]phenyl}-N′-(5-cyanopyridin-2-yl)pyrazine-2,3-dicarboxamide

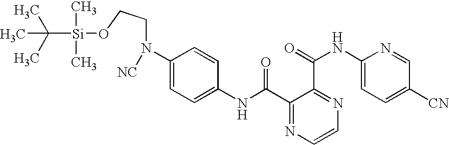

At RT, 56 mg (66 mmol) of sodium bicarbonate and 80 μl (0.24 mmol) of a 3 M solution of cyanogen bromide in dichloromethane are added to a solution of 114 mg (0.22 mmol) of N-{4-[(2-{[tert-butyl(dimethyl)silyl]oxy}ethyl)amino]phenyl}-N′-(5-cyanopyridin-2-yl)pyrazine-2,3-dicarboxamide in 2 ml of THF. The suspension is stirred at 40° C. overnight, a further 16 μl (0.04 mmol) of a 3 M solution of cyanogen bromide in dichloromethane are then added and the mixture is again stirred at 40° C. overnight. After addition of 2 ml of water/4 ml of dichloromethane and phase separation, the organic phase is washed with saturated aqueous sodium bicarbonate solution, dried over magnesium sulphate, filtered and concentrated under reduced pressure. The residue is triturated in diisopropyl ether and filtered, and the solid is dried under reduced pressure.

Yield: 90 mg (91% pure, 67% of theory)

LC-MS (method 3): Rₜ=2.40 min;

MS (ESIpos): m/z=543 [M+H]⁺.

### Step c): N-(5-Cyanopyridin-2-yl)-N′-[4-(2-imino-1,3-oxazolidin-3-yl)phenyl]-pyrazine-2,3-dicarboxamide methanesulphonate

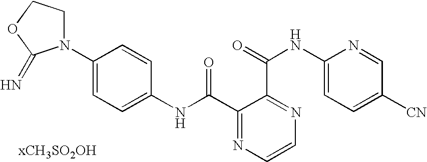

At RT, 15 μl (0.24 mmol) of methanesulphonic acid are added to a solution of 61 mg (0.11 mmol) of N-{4-[(2-{[tert-butyl(dimethyl)silyl]oxy}ethyl)(cyano)amino]phenyl}-N′-(5-cyanopyridin-2-yl)pyrazine-2,3-dicarboxamide in 5 ml of acetonitrile, and the mixture is stirred at RT overnight. The reaction mixture is then concentrated under reduced pressure and the residue is dried.

Yield: 60 mg (quant.)

LC-MS (method 6): Rₜ=1.23 min;

MS (ESIpos): m/z=429 [M+H]⁺ (free base);

¹H NMR (400 MHz, DMSO-d₆): δ=11.49 (s, 1H), 11.11 (s, 1H), 9.57 (s, 1H), 8.97 (s, 2H), 8.82 (s, 2H), 8.34 (s, 2H), 7.98 (d, 2H), 7.51 (d, 2H), 4.85 (t, 2H), 4.23 (t, 2H), 2.32 (s, 3H).

### Example 8

### N-(4-Cyanophenyl)-N′-[4-(2-imino-1,3-oxazolidin-3-yl)phenyl]pyrazine-2,3-dicarboxamide methanesulphonate

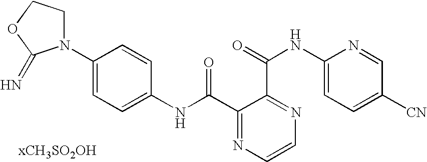

### Step a): N-{4-[(2-{[tert-Butyl(dimethyl)silyl]oxy}ethyl)amino]phenyl}-N′-(4-cyanophenyl)pyrazine-2,3-dicarboxamide

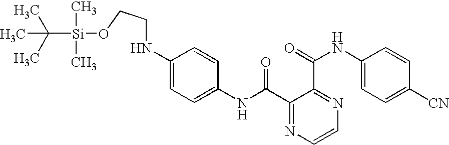

According to the General Method 1, 500 mg (1.86 mmol) of the compound from Example 6A are reacted with 497 mg (1.86 mmol) of the compound from Example 1A.

Yield: 437 mg (45% of theory)

LC-MS (method 2): Rₜ=2.89 min;

MS (ESIpos): m/z=517 [M+H]⁺.

### Step b): N-{4-[(2-{[tert-Butyl(dimethyl)silyl]oxy}ethyl)(cyano)amino]phenyl}-N′-(4-cyanophenyl)pyrazine-2,3-dicarboxamide

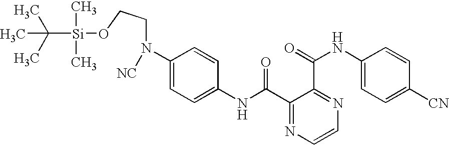

At RT, 98 mg (1.16 mmol) of sodium bicarbonate and 143 μl (0.43 mmol) of a 3 M solution of cyanogen bromide in dichloromethane are added to a solution of 200 mg (0.39 mmol) of N-{4-[(2-{[tert-butyl(dimethyl)silyl]oxy}ethyl)amino]phenyl}-N′-(4-cyanophenyl)pyrazine-2,3-dicarboxamide in 2 ml of THF. The suspension is stirred at 40° C. overnight, and then diluted with 3 ml of water and 5 ml of dichloromethane. After phase separation, the organic phase is washed with saturated aqueous sodium bicarbonate solution, dried over magnesium sulphate, filtered and concentrated under reduced pressure.

Yield: 159 mg (76% of theory)

LC-MS (method 1): Rₜ=2.57 min;

MS (ESIpos): m/z=542 [M+H]⁺;

¹H NMR (300 MHz, DMSO-d₆): δ=11.18 (s, 1H), 10.84 (s, 1H), 8.99 (s, 2H), 7.97 (d, 2H), 7.86 (d, 2H), 7.82 (d, 2H), 7.22 (d, 2H), 3.90-3.79 (m, 4H), 0.86 (s, 9H), 0.0 (s, 6H).

### Step c): N-(4-Cyanophenyl)-N′-[4-(2-imino-1,3-oxazolidin-3-yl)phenyl]pyrazine-2,3-dicarboxamide methanesulphonate

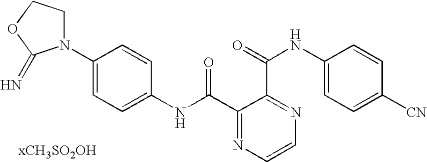

At RT, 13 μl (0.19 mmol) of methanesulphonic acid are added to a solution of 50 mg (0.09 mmol) of N-{4-[(2-{[tert-butyl(dimethyl)silyl]oxy}ethyl)(cyano)amino]phenyl}-N′-(4-cyanophenyl)pyrazine-2,3-dicarboxamide in 5 ml of acetonitrile, and the mixture is stirred at RT overnight. The reaction mixture is then concentrated under reduced pressure and the residue is dried.

Yield: 47 mg (97% of theory)

LC-MS (method 3): Rₜ=1.20 min;

MS (ESIpos): m/z=428 [M+H]⁺ (free base);

¹H NMR (300 MHz, DMSO-d₆): δ=11.22 (s, 1H), 11.06 (s, 1H), 9.58 (s, 1H), 9.00 (s, 2H), 8.81 (s, 1H), 7.99-7.88 (m, 4H), 7.84 (d, 2H), 7.51 (d, 2H), 4.85 (t, 2H), 4.23 (t, 2H), 2.31 (s, 3H).

### Example 9

### N-(4-Ethynylphenyl)-N′-[4-(2-imino-1,3-oxazolidin-3-yl)phenyl]pyrazine-2,3-dicarboxamide methanesulphonate

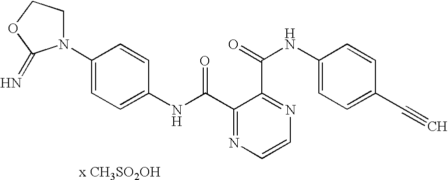

### Step a): N-{4-[(2-{[tert-Butyl(dimethyl)silyl]oxy}ethyl)amino]phenyl}-N′-(4-ethynylphenyl)pyrazine-2,3-dicarboxamide

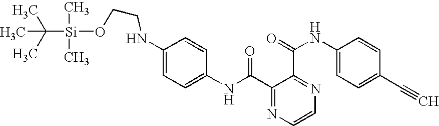

According to the General Method 1, 400 mg (1.50 mmol) of the compound from Example 7A are reacted with 398 mg (1.50 mmol) of the compound from Example 1A.

Yield: 376 mg (49% of theory)

LC-MS (method 2): Rₜ=3.08 min;

MS (ESIpos): m/z=516 [M+H]⁺.

### Step b): N-{4-[(2-{[tert-Butyl(dimethyl)silyl]oxy}ethyl)(cyano)amino]phenyl}-N′-(4-ethynylphenyl)pyrazine-2,3-dicarboxamide

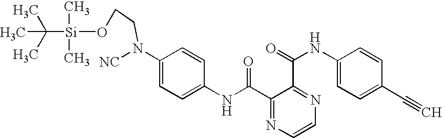

At RT, 73 mg (0.87 mmol) of sodium bicarbonate and 107 μl (0.32 mmol) of a 3 M solution of cyanogen bromide in dichloromethane are added to a solution of 150 mg (0.29 mmol) of N-{4-[(2-{[tert-butyl(dimethyl)silyl]oxy}ethyl)amino]phenyl}-N′-(4-ethynylphenyl)pyrazine-2,3-dicarboxamide in 2 ml of THF. The suspension is stirred at 40° C. for 15 h and then diluted with 1.5 ml of water and 2 ml of dichloromethane. After phase separation, the organic phase is washed with saturated aqueous sodium bicarbonate solution, dried over magnesium sulphate, filtered and concentrated under reduced pressure. The residue is triturated with diisopropyl ether and filtered, and the solid is dried under reduced pressure.

Yield: 65 mg (41% of theory)

LC-MS (method 3): Rₜ=2.61 min;

MS (ESIpos): m/z=541 [M+H]⁺;

¹H NMR (400 MHz, DMSO-d₆): δ=10.90 (s, 1H), 10.79 (s, 1H), 8.94 (s, 2H), 7.82-7.72 (m, 4H), 7.46 (d, 2H), 7.19 (d, 2H), 4.12 (s, 1H), 3.86-3.74 (m, 4H), 0.82 (s, 9H), 0.0 (s, 6H).

### Step c): N-(4-Ethynylphenyl)-N′-[4-(2-imino-1,3-oxazolidin-3-yl)phenyl]pyrazine-2,3-dicarboxamide methanesulphonate

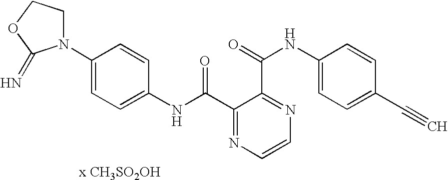

At RT, 13 μl (0.19 mmol) of methanesulphonic acid are added to a solution of 50 mg (0.09 mmol) of N-{4-[(2-{[tert-butyl(dimethyl)silyl]oxy}ethyl)(cyano)amino]phenyl}-N′-(4-ethynylphenyl)pyrazine-2,3-dicarboxamide in 10 ml of acetonitrile, and the mixture is stirred at RT overnight. The precipitate formed is filtered off, washed with acetonitrile and dried under reduced pressure.

Yield: 18 mg (37% of theory)

LC-MS (method 3): Rₜ=1.47 min;

MS (ESIpos): m/z=427 [M+H]⁺ (free base);

¹H NMR (300 MHz, DMSO-d₆): δ=11.02 (s, 1H), 10.97 (s, 1H), 9.57 (s, 1H), 8.98 (s, 2H), 8.81 (s, 1H), 7.92 (d, 2H), 7.78 (d, 2H), 7.55-7.44 (m, 4H), 4.85 (t, 2H), 4.23 (t, 2H), 4.15 (s, 1H), 2.29 (s, 3H).

### Example 10

### N³-(5-Chloropyridin-2-yl)-N²-[4-(2-imino-1,3-oxazolidin-3-yl)phenyl]thiophene-2,3-dicarboxamide methanesulphonate

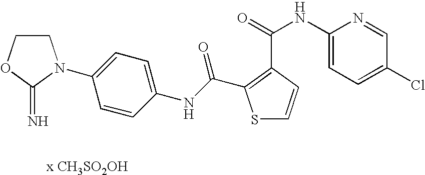

### Step a): N²-{4-[(2-{[tert-Butyl(dimethyl)silyl]oxy}ethyl)amino]phenyl}-N³-(5-chloropyridin-2-yl)thiophene-2,3-dicarboxamide

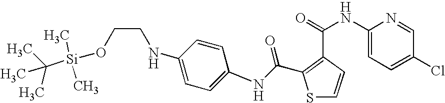

According to the General Method 1, 928 mg (2.95 mmol) of the mixture of regioisomers from Example 8A are reacted with 787 mg (2.95 mmol) of the compound from Example 1A. The crude product is purified by preparative RP-HPLC [column: YMC-Pack Polyamine II, 5 μm, 250 mm×20 mm; mobile phase: ethanol/isohexane 1:4], resulting in the separation of the two regioisomeric products (cf. also Example 11).

Yield: 370 mg (24% of theory) LC-MS (method 3): Rₜ=3.20 min;

MS (ESIpos): m/z=531 [M+H]⁺;

¹H NMR (300 MHz, DMSO-d₆): δ=11.63 (s, 1H), 10.89 (s, 1H), 8.43 (d, 1H), 8.20 (d, 1H), 7.97 (dd, 1H), 7.81 (d, 1H), 7.67 (d, 1H), 7.35 (d, 2H), 6.58 (d, 2H), 5.50 (t, 1H), 3.69 (t, 2H), 3.12 (qd, 2H), 0.85 (s, 9H), 0.03 (s, 6H).

### Step b): N²-{4-[(2-{[tert-Butyl(dimethyl)silyl]oxy}ethyl)(cyano)amino]phenyl}-N³-(5-chloropyridin-2-yl)thiophene-2,3-dicarboxamide

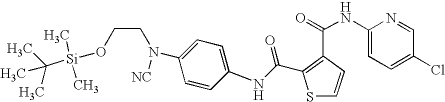

At RT, 175 mg (2.09 mmol) of sodium bicarbonate and 280 μl (0.84 mmol) of a 3 M solution of cyanogen bromide in dichloromethane are added to a solution of 370 mg (0.70 mmol) of N²-{4-[(2-{[tert-butyl(dimethyl)silyl]oxy}ethyl)amino]phenyl}-N-3-(5-chloropyridin-2-yl)thiophene-2,3-dicarboxamide in 3 ml of THF. The suspension is stirred at 40° C. for 2 d and then diluted with 6 ml of water and 10 ml of dichloromethane. After phase separation, the organic phase is washed with saturated aqueous sodium bicarbonate solution, dried over magnesium sulphate, filtered and concentrated under reduced pressure. The residue is triturated with diethyl ether and filtered, and the solid is dried under reduced pressure.

Yield: 275 mg (71% of theory)

LC-MS (method 3): Rₜ=3.27 min;

MS (ESIpos): m/z=556 [M+H]⁺;

¹H NMR (400 MHz, DMSO-d₆): δ=11.48 (s, 1H), 11.19 (s, 1H), 8.47 (d, 1H), 8.23 (d, 1H), 8.00 (dd, 1H), 7.90 (d, 1H), 7.73 (d, 1H), 7.71 (d, 2H), 7.22 (d, 2H), 3.85 (br. s, 4H), 0.86 (s, 9H), 0.0 (s, 6H).

### Step c): N³-(5-Chloropyridin-2-yl)-N²-[4-(2-imino-1,3-oxazolidin-3-yl)-phenyl]thiophene-2,3-dicarboxamide methanesulphonate

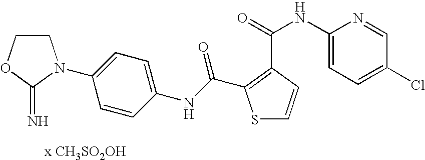

At RT, 70 μl (1.04 mmol) of methanesulphonic acid are added to a solution of 275 mg (0.49 mmol) of N²-{4-[(2-{[tert-butyl(dimethyl)silyl]oxy}ethyl)(cyano)amino]phenyl}-N³-(5-chloropyridin-2-yl)thiophene-2,3-dicarboxamide in 37 ml of acetonitrile, and the mixture is stirred at RT for 4 d. The precipitate formed is filtered off, washed with acetonitrile and dried under reduced pressure.

Yield: 230 mg (87% of theory)

HPLC (method 7): Rₜ=4.01 min;

MS (ESIpos): m/z=442 [M+H]⁺ (free base);

¹H NMR (400 MHz, DMSO-d₆): δ=11.38 (s, 1H), 11.34 (s, 1H), 9.58 (br. s, 1H), 8.81 (br. s, 1H), 8.45 (d, 1H), 8.19 (d, 1H), 7.99 (dd, 1H), 7.90 (d, 1H), 7.82 (d, 2H), 7.73 (d, 1H), 7.52 (d, 2H), 4.86 (t, 2H), 4.23 (t, 2H), 2.32 (s, 3H).

### Example 11

### N²-(5-Chloropyridin-2-yl)-N-3-[4-(2-imino-1,3-oxazolidin-3-yl)phenyl]thiophene-2,3-dicarboxamide methanesulphonate

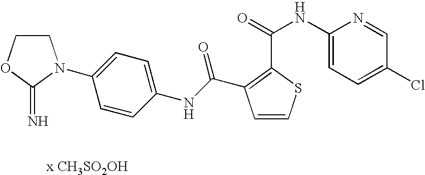

### Step a): N³-{4-[(2-{([tert-Butyl(dimethyl)silyl]oxy}ethyl)amino]phenyl}-N²-(5-chloropyridin-2-yl)thiophene-2,3-dicarboxamide

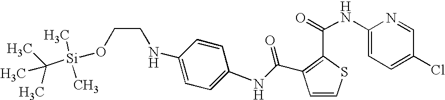

According to the General Method 1, 928 mg (2.95 mmol) of the mixture of regioisomers from Example 8A are reacted with 787 mg (2.95 mmol) of the compound from Example 1A. The crude product is purified by preparative RP-HPLC [column: YMC-Pack Polyamine II, 5 μm, 250 mm×20 mm; mobile phase: ethanol/isohexane 1:4], resulting in the separation of the two regioisomeric products (cf. also Example 10).

Yield: 188 mg (12% of theory)

LC-MS (method 3): Rₜ=3.22 min;

MS (ESIpos): m/z=531 [M+H]⁺;

¹H NMR (300 MHz, DMSO-d₆): δ=13.11 (s, 1H), 10.40 (s, 1H), 8.40 (d, 1H), 8.21 (d, 1H), 8.00 (d, 1H), 7.95 (dd, 1H), 7.71 (d, 1H), 7.39 (d, 2H), 6.60 (d, 2H), 5.58 (t, 1H), 3.70 (t, 2H), 3.14 (qd, 2H), 0.86 (s, 9H), 0.03 (s, 6H).

### Step b): N³-{4-[(2-{[tert-Butyl(dimethyl)silyl]oxy}ethyl)(cyano)amino]phenyl}-N²-(5-chloropyridin-2-yl)thiophene-2,3-dicarboxamide

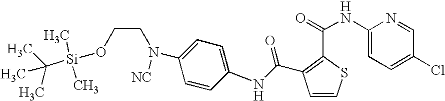

At RT, 89 mg (1.06 mmol) of sodium bicarbonate and a total of 160 μl (0.49 mmol) of a 3 M solution of cyanogen bromide in dichloromethane are added to a solution of 188 mg (0.35 mmol) of N³-{4-[(2-{[tert-butyl(dimethyl)silyl]oxy}ethyl)amino]phenyl}-N²-(5-chloropyridin-2-yl)thiophene-2,3-dicarboxamide in 3 ml of THF. The suspension is stirred at 40° C. for 2 d and then diluted with 3 ml of water and 4 ml of dichloromethane. After phase separation, the organic phase is washed with saturated aqueous sodium bicarbonate solution, dried over magnesium sulphate, filtered and concentrated under reduced pressure. The residue is triturated in diisopropyl ether and filtered, and the solid is dried under reduced pressure.

Yield: 146 mg (74% of theory)

LC-MS (method 2): Rₜ=3.33 min;

MS (ESIpos): m/z=556 [M+H]⁺;

¹H NMR (400 MHz, DMSO-d₆): δ=12.73 (s, 1H), 10.72 (s, 1H), 8.43 (d, 1H), 8.23 (d, 1H), 8.04 (d, 1H), 7.99 (dd, 1H), 7.81-7.60 (2d, 3H), 7.27 (d, 2H), 3.85 (br. s, 4H), 0.85 (s, 9H), 0.01 (s, 6H).

### Step c): N²-(5-Chloropyridin-2-yl)-N³-[4-(2-imino-1,3-oxazolidin-3-yl)phenyl]thiophene-2,3-dicarboxamide methanesulphonate

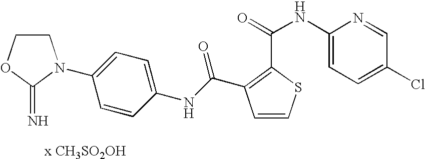

At RT, 40 μl (0.55 mmol) of methanesulphonic acid are added to a solution of 146 mg (0.26 mmol) of N³-{4-[(2-{[tert-butyl(dimethyl)silyl]oxy}ethyl)(cyano)amino]phenyl}-N²-(5-chloropyridin-2-yl)thiophene-2,3-dicarboxamide in 50 ml of acetonitrile, and the mixture is stirred at RT for 3 d. The precipitate formed is filtered off, washed with acetonitrile and dried under reduced pressure. The mother liquor is purified by flash chromatography on silica gel (mobile phase: dichloromethane/methanol 8:1→4:1).

Yield: 131 mg in total (93% of theory)

HPLC (method 7): Rₜ=4.18 min;

MS (ESIpos): m/z=442 [M+H]⁺ (free base);

¹H NMR (300 MHz, DMSO-d₆): δ=12.53 (s, 1H), 10.91 (s, 1H), 9.44 (br. s, 1H), 8.95 (br. s, 1H), 8.42 (d, 1H), 8.22 (d, 1H), 8.07 (d, 1H), 7.99 (dd, 1H), 7.88 (d, 2H), 7.74 (d, 1H), 7.57 (d, 2H), 4.84 (t, 2H), 4.23 (t, 2H), 2.30 (s, 3H).

### Example 12

### N⁴-(5-Chloropyridin-2-yl)-N⁵-[4-(2-imino-1,3-oxazolidin-3-yl)phenyl]-2-methyl-1,3-thiazole-4,5-dicarboxamide methanesulphonate

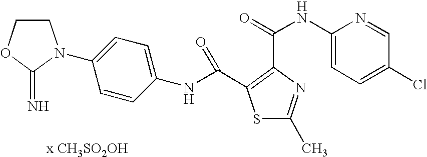

### Step a): N⁵-{4-[(2-{[tert-Butyl(dimethyl)silyl]oxy}ethyl)amino]phenyl}-N⁴-(5-chloropyridin-2-yl)-2-methyl-1,3-thiazole-4,5-dicarboxamide

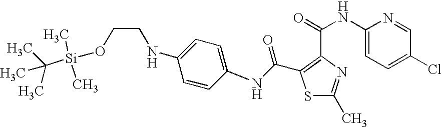

According to the General Method 1, the mixture of regioisomers from Example 9A (as crude product, about 27 mmol) is reacted with 7.2 g (27 mmol) of the compound from Example 1A. The crude product obtained is purified by preparative RP-HPLC [column: Kromasil 100 C18, 5 μm, 250 mm×20 mm; mobile phase: water/acetonitrile 1:9], resulting in the separation of the two regioisomeric products (cf. also Example 13).

Yield: 2.5 g (17% of theory)

LC-MS (method 1): Rₜ=3.46 min;

MS (ESIpos): m/z=546 [M+H]⁺;

¹H NMR (400 MHz, DMSO-d₆): δ=12.16 (s, 1H), 10.42 (s, 1H), 8.50 (d, 1H), 8.25 (d, 1H), 8.05 (dd, 1H), 7.41 (d, 2H), 6.62 (d, 2H), 5.54 (t, 1H), 3.71 (t, 2H), 3.15 (qd, 2H), 2.76 (s, 3H), 0.88 (s, 9H), 0.03 (s, 6H).

### Step b): N⁵-{4-[(2-{[tert-Butyl(dimethyl)silyl]oxy}ethyl)(cyano)amino]phenyl}-N⁴-(5-chloropyridin-2-yl)-2-methyl-1,3-thiazole-4,5-dicarboxamide

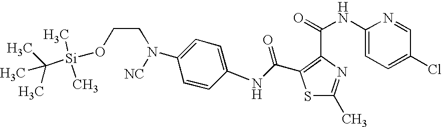

At RT, 503 mg (6.0 mmol) of sodium bicarbonate and a total of 1.1 ml (3.2 mmol) of a 3 M solution of cyanogen bromide in dichloromethane are added to a solution of 1.09 g (2.0 mmol) of N⁵-{4-[(2-{[tert-butyl(dimethyl)silyl]oxy}ethyl)amino]phenyl}-N⁴-(5-chloropyridin-2-yl)-2-methyl-1,3-thiazole-4,5-dicarboxamide in 17 ml of THF. The suspension is stirred at 40° C. for 4 d and then diluted with 17 ml of water and 23 ml of dichloromethane. After phase separation, the organic phase is washed with saturated aqueous sodium bicarbonate solution, dried over magnesium sulphate, filtered and concentrated under reduced pressure. The residue is triturated with diisopropyl ether and filtered, and the solid is dried under reduced pressure.

Yield: 1.19 g (quant.)

HPLC (method 8): Rₜ=6.19 min;

MS (ESIpos): m/z=571 [M+H]⁺;

¹H NMR (400 MHz, DMSO-d₆): δ=12.30 (s, 1H), 10.46 (s, 1H), 8.51 (d, 1H), 8.27 (d, 1H), 8.09 (dd, 1H), 7.74 (d, 2H), 7.26 (d, 2H), 3.86 (m, 4H), 0.86 (s, 9H), 0.0 (s, 6H).

### Step c): N⁴-(5-Chloropyridin-2-yl)-N⁵-[4-(2-imino-1,3-oxazolidin-3-yl)phenyl]-2-methyl-1,3-thiazole-4,5-dicarboxamide methanesulphonate

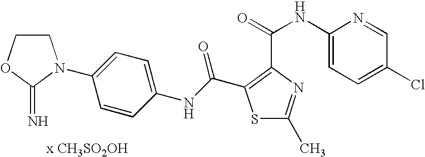

At RT, 329 μl (5.07 mmol) of methanesulphonic acid are added to a solution of 1.38 g (2.42 mmol) of N⁵-{4-[(2-{[tert-butyl(dimethyl)silyl]oxy}ethyl)(cyano)amino]phenyl}-N-(5-chloropyridin-2-yl)-2-methyl-1,3-thiazole-4,5-dicarboxamide in 450 ml of acetonitrile, and the mixture is stirred at RT for 5 d. The precipitate formed is filtered off, washed with acetonitrile and dried under reduced pressure.

Yield: 1.33 g (98% of theory)

HPLC (method 7): Rₜ=4.33 min;

MS (ESIpos): m/z=457 [M+H]⁺ (free base);

¹H NMR (400 MHz, DMSO-d₆): δ=12.37 (s, 1H), 10.45 (s, 1H), 9.58 (br. s, 1H), 8.83 (br. s, 1H), 8.50 (s, 1H), 8.21 (d, 1H), 8.07 (d, 1H), 7.85 (d, 2H), 7.56 (d, 2H), 4.86 (t, 2H), 2.80 (s, 3H), 2.31 (s, 3H).

### Example 13

### N⁵-(5-Chloropyridin-2-yl)-N⁴-[4-(2-imino-1,3-oxazolidin-3-yl)phenyl]-2-methyl-1,3-thiazole-4,5-dicarboxamide methanesulphonate

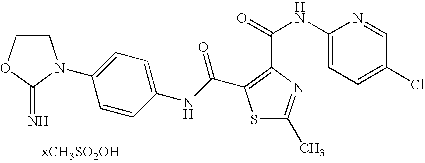

### Step a): N⁴-{4-[(2-{[tert-Butyl(dimethyl)silyl]oxy}ethyl)amino]phenyl}-N⁵-(5-chloropyridin-2-yl)-2-methyl-1,3-thiazole-4,5-dicarboxamide

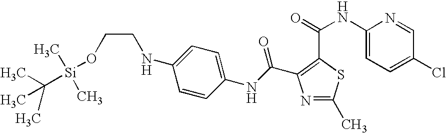

According to the General Method 1, the mixture of regioisomers from Example 9A (as crude product, about 27 mmol) is reacted with 7.2 g (27 mmol) of the compound from Example 1A. The crude product obtained is purified by preparative RP-HPLC [column: Kromasil 100 C18, 5 μm, 250 mm×20 mm; mobile phase: water/acetonitrile 1:9], resulting in the separation of the two regioisomeric products (cf. also Example 12).

Yield: 2.38 g (16% of theory)

HPLC (method 8): Rₜ=4.97 min;

MS (ESIpos): m/z=546 [M+H]⁺;

¹H NMR (400 MHz, DMSO-d₆): δ=13.90 (s, 1H), 10.41 (s, 1H), 8.40 (d, 1H), 8.18 (d, 1H), 7.93 (dd, 1H), 7.46 (d, 2H), 6.58 (d, 2H), 5.55 (t, 1H), 3.68 (t, 2H), 3.11 (qd, 2H), 2.71 (s, 3H), 0.83 (s, 9H), 0.0 (s, 6H).

### Step b): N⁴-{4-[(2-{[tert-Butyl(dimethyl)silyl]oxy}ethyl)(cyano)amino]phenyl}-N⁵-(5-chloropyridin-2-yl)-2-methyl-1,3-thiazole-4,5-dicarboxamide

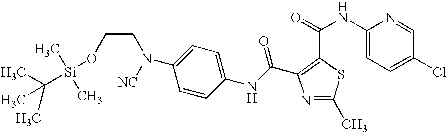

At RT, 68 mg (0.81 mmol) of sodium bicarbonate and a total of 144 μl (0.43 mmol) of a 3 M solution of cyanogen bromide in dichloromethane are added to a solution of 147 mg (0.27 mmol) of N⁴-{4-[(2-{[tert-butyl(dimethyl)silyl]oxy}ethyl)amino]phenyl}-N⁵-(5-chloropyridin-2-yl)-2-methyl-1,3-thiazole-4,5-dicarboxamide in 2.5 ml of THF. The suspension is stirred at 40° C. for 4 d and then diluted with 2.5 ml of water and 3.5 ml of dichloromethane. After phase separation, the organic phase is washed with saturated aqueous sodium bicarbonate solution, dried over magnesium sulphate, filtered and concentrated under reduced pressure. The residue is purified by flash chromatography on silica gel (mobile phase: dichloromethane/methanol 200:1).

Yield: 84 mg (49% of theory)

HPLC (method 8): Rₜ=5.86 min;

MS (ESIpos): m/z=571 [M+H]⁺;

¹H NMR (400 MHz, DMSO-d₆): δ=13.59 (s, 1H), 10.84 (s, 1H), 8.46 (d, 1H), 8.23 (d, 1H), 8.00 (dd, 1H), 7.87 (d, 2H), 7.26 (d, 2H), 3.85 (br. s, 4H), 2.79 (s, 3H), 0.85 (s, 9H), 0.0 (s, 6H).

### Step c): N⁵-(5-Chloropyridin-2-yl)-N⁴-[4-(2-imino-1,3-oxazolidin-3-yl)phenyl]-2-methyl-1,3-thiazole-4,5-dicarboxamide methanesulphonate

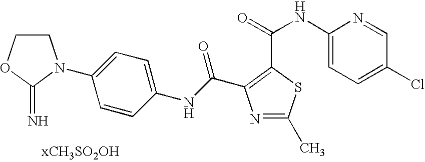

At RT, 20 μl (0.31 mmol) of methanesulphonic acid are added to a solution of 83 mg (0.15 mmol) of N-({4-[(2-{[tert-butyl(dimethyl)silyl]oxy}ethyl)(cyano)amino]phenyl}-N⁵-(5-chloropyridin-2-yl)-2-methyl-1,3-thiazole-4,5-dicarboxamide in 26 ml of acetonitrile, and the mixture is stirred at RT for 1 d. The reaction mixture is concentrated under reduced pressure and the residue is triturated in acetonitrile/dichloromethane, filtered off and dried under reduced pressure.

Yield: 29 mg (36% of theory)

HPLC (method 9): Rₜ=4.37 min;

MS (ESIpos): m/z=457 [M+H]⁺ (free base);

¹H NMR (400 MHz, DMSO-d₆): δ=13.40 (s, 1H), 11.04 (s, 1H), 9.62 (br. s, 1H), 8.92 (br. s, 1H), 8.47 (d, 1H), 8.23 (d, 1H), 8.03 (d, 1H), 8.00 (d, 2H), 7.58 (d, 2H), 4.87 (t, 2H), 4.28 (t, 2H), 2.80 (s, 3H), 2.30 (s, 3H).

### Example 14

### N⁴-(5-Chloropyridin-2-yl)-N⁵-[4-(2-imino-1,3-oxazolidin-3-yl)phenyl]-1H-imidazole-4,5-dicarboxamide methanesulphonate

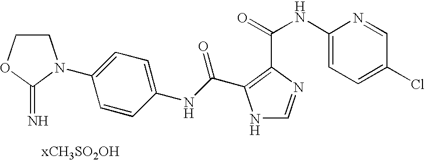

### Step a): Methyl 5-[({4-[(2-{[tert-butyl(dimethyl)silyl]oxy}ethyl)amino]-phenyl}amino)carbonyl]-1H-imidazole-4-carboxylate

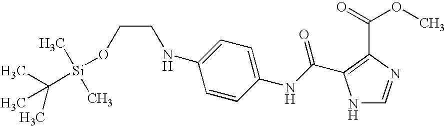

Under argon and at RT, a solution of 1.57 g (5.9 mmol) of the compound from Example 1A in 5 ml of THF is added over a period of 2 min to a suspension of 895 mg (2.94 mmol) of the compound from Example 11A in 45 ml of THF. The reaction mixture is stirred at RT overnight, the THF is then removed under reduced pressure and the residue is taken up in dichloromethane. This solution is washed with saturated aqueous sodium bicarbonate solution, dried over sodium sulphate, filtered and concentrated under reduced pressure. The residue is used without further purification for the next step.

Yield: 2.2 g (87% pure, 77% of theory)

LC-MS (method 1): Rₜ=2.46 min;

MS (ESIpos): m/z=419 [M+H]⁺.

### Step b): N⁵-{4-[(2-{[tert-Butyl(dimethyl)silyl]oxy}ethyl)amino]phenyl}-N⁴-(5-chloropyridin-2-yl)-1H-imidazole-4,5-dicarboxamide

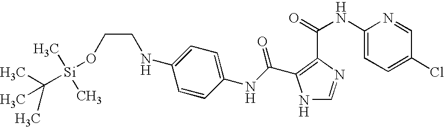

Under argon and at 0° C., 540 μl of trimethylaluminium solution (2 M in hexane, 1.08 mmol) are added dropwise to a solution of 55 mg (0.43 mmol) of 2-amino-5-chloropyridine in 2 ml of dichloromethane. The reaction mixture is allowed to warm to RT, stirred at RT for 15 min and again cooled to 0° C., and a solution of 90 mg (0.22 mmol) of methyl 5-[({4-[(2-{[tert-butyl(dimethyl)silyl]oxy}ethyl)amino]phenyl}amino)carbonyl]-1H-imidazole-4-carboxylate is then added. The reaction mixture is stirred at RT, and a 20% strength potassium tartrate solution is then added dropwise (careful: vigorous foaming!). After addition of dichloromethane and phase separation, the organic phase is washed with water, dried over sodium sulphate, filtered and concentrated under reduced pressure. The crude product is purified by preparative RP-HPLC.

Yield: 20 mg (18% of theory)

LC-MS (method 1): Rₜ=3.15 min;

MS (ESIpos): m/z=515 [M+H]⁺.

### Step c): N⁵-{4-[(2-{[tert-Butyl(dimethyl)silyl]oxy}ethyl)(cyano)amino]phenyl}-N⁴-(5-chloropyridin-2-yl)-1H-imidazole-4,5-dicarboxamide

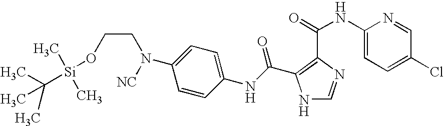

At RT, 37 mg (0.44 mmol) of sodium bicarbonate and a total of 100 μl (0.32 mmol) of a 3 M solution of cyanogen bromide in dichloromethane are added to a solution of 75 mg (0.15 mmol) of N⁵-{4-[(2-{[tert-butyl(dimethyl)silyl]oxy}ethyl)amino]phenyl}-N⁴-(5-chloropyridin-2-yl)-1H-imidazole-4,5-dicarboxamide in 2.3 ml of THF. The suspension is stirred at 40° C. for 2 d. The precipitate formed is filtered off, washed with THF and dried under reduced pressure.

Yield: 14 mg (18% of theory)

LC-MS (method 1): Rₜ=3.05 min;

MS (ESIpos): m/z=540 [M+H]⁺.

### Step d): N⁴-(5-Chloropyridin-2-yl)-N⁵-[4-(2-imino-1,3-oxazolidin-3-yl)phenyl]-1H-imidazole-4,5-dicarboxamide methanesulphonate

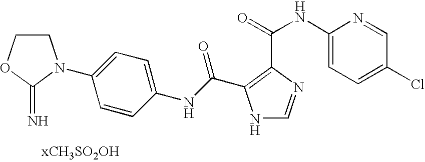

At RT, 4 μl (0.07 mmol) of methanesulphonic acid are added to a solution of 17 mg (0.03 mmol) of N⁵-{4-[(2-{[tert-butyl(dimethyl)silyl]oxy}ethyl)(cyano)amino]phenyl}-N⁴-(5-chloropyridin-2-yl)-1H-imidazole-4,5-dicarboxamide in 5 ml of acetonitrile, and the mixture is stirred at RT for 1 d. The reaction mixture is concentrated under reduced pressure and the residue is triturated in a diethyl ether/methanol/acetonitrile mixture, filtered off and dried under reduced pressure.

Yield: 12 mg (77% of theory)

LC-MS (method 3): Rₜ=1.38 min;

MS (ESIpos): m/z=426 [M+H]⁺ (free base);

¹H NMR (400 MHz, DMSO-d₆): δ=13.06 (s, 1H), 11.13 (s, 1H), 9.60 (br. s, 1H), 8.88 (br. s, 1H), 8.47 (d, 1H), 8.32 (d, 1H), 8.13 (s, 1H), 8.03 (d, 3H), 7.58 (d, 2H), 4.87 (t, 1H), 4.28 (t, 2H), 2.34 (s, 3H).

### Example 15

### N⁴-(4-Chlorophenyl)-N⁵-[4-(2-imino-1,3-oxazolidin-3-yl)phenyl]-1H-imidazole-4,5-dicarboxamide methanesulphonate

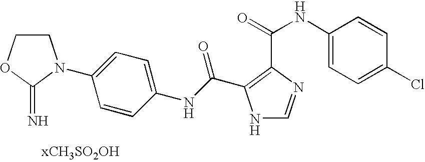

### Step a): Phenyl 5-[({4-[(2-{[tert-butyl(dimethyl)silyl]oxy}ethyl)amino]-phenyl}amino)carbonyl]-1H-imidazole-4-carboxylate

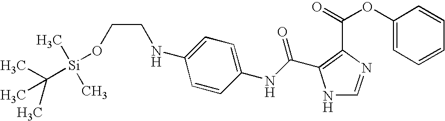

Under argon and at RT, a solution of 1.1 g (4.14 mmol) of the compound from Example 1A in 3 ml of THF is added over a period of 2 min to a suspension of 887 mg (2.07 mmol) of the compound from Example 10A in 12 ml of THF. The reaction mixture is stirred at RT overnight, the THF is then removed under reduced pressure and the residue is taken up in dichloromethane. This solution is washed with saturated aqueous sodium bicarbonate solution, dried over sodium sulphate, filtered and concentrated under reduced pressure. The residue is used without further purification for the next step.

Yield: 1.95 g (98% of theory)

LC-MS (method 3): Rₜ=2.97 min;

MS (ESIpos): m/z=481 [M+H]⁺.

### Step b): 5-[({4-[(2-{[tert-Butyl(dimethyl)silyl]oxy}ethyl)amino]phenyl}-amino)carbonyl]-1H-imidazole-4-carboxylic acid

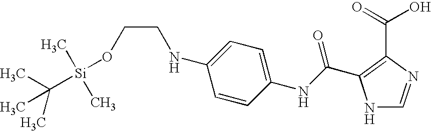

At RT, 194 mg (8.11 mmol) of lithium hydroxide are added to a solution of 1.95 g (4.06 mmol) of phenyl 5-[({4-[(2-{[tert-butyl(dimethyl)silyl]oxy}ethyl)amino]-phenyl}amino)carbonyl]-1H-imidazole-4-carboxylate in 97.5 ml of THF/water (3:1), and the mixture is stirred at 60° C. overnight. The THF is removed under reduced pressure and the residue is acidified to pH 1 using 1 N hydrochloric acid. The resulting precipitate is filtered off, washed with water and dried under reduced pressure.

Yield: 1.65 g (90% of theory)

LC-MS (method 3): Rₜ=2.49 min;

MS (ESIpos): m/z=405 [M+H]⁺.

### Step c): N⁵-{4-[(2-{[tert-Butyl(dimethyl)silyl]oxy}ethyl)amino]phenyl}-N⁴-(4-chlorophenyl)-1H-imidazole-4,5-dicarboxamide

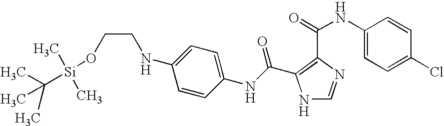

At RT, 56 μl (0.32 mmol) of N,N-diisopropylethylamine, 32 mg (0.25 mmol) of 4-chloroaniline and 122 mg (0.32 mmol) of O-(7-azabenzotriazol-1-yl)-N,N,N′,N′-tetramethyluronium hexafluorophosphate (HATU) are added to a solution of 100 mg (0.25 mmol) of 5-[({4-[(2-{[tert-butyl(dimethyl)silyl]oxy}ethyl)amino]phenyl}-amino)carbonyl]-1H-imidazole-4-carboxylic acid in 2 ml of THF and 0.5 ml of dimethylformamide. The reaction mixture is stirred at RT overnight, the THF is then removed under reduced pressure and the crude product is purified by preparative RP-HPLC.

Yield: 63 mg (50% of theory)

LC-MS (method 3): Rₜ=3.39 min;

MS (ESIpos): m/z=514 [M+H]⁺.

### Step d): N⁵-{4-[(2-{[tert-Butyl(dimethyl)silyl]oxy}ethyl)(cyano)amino]phenyl}-N⁴-(4-chlorophenyl)-1H-imidazole-4,5-dicarboxamide

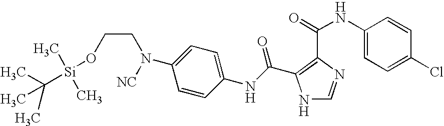

At RT, 29 mg (0.35 mmol) of sodium bicarbonate and a total of 70 μl (0.21 mmol) of a 3 M solution of cyanogen bromide in dichloromethane are added to a solution of 60 mg (0.12 mmol) of N⁵-{4-[(2-{[tert-butyl(dimethyl)silyl]oxy}ethyl)amino]phenyl}-4-(4-chlorophenyl)-1H-imidazole-4,5-dicarboxamide in 5 ml of THF. The suspension is stirred at 40° C. for 2 d and then diluted with 3 ml of water and 5 ml of dichloromethane. After phase separation, the organic phase is washed with saturated aqueous sodium bicarbonate solution, dried over sodium sulphate, filtered and concentrated under reduced pressure. The residue is purified by preparative RP-HPLC.

Yield: 21 mg (33% of theory)

LC-MS (method 1): Rₜ=3.13 min;

MS (ESIpos): m/z=539 [M+H]⁺.

### Step e): N⁴-(4-Chlorophenyl)-N⁵-[4-(2-imino-1,3-oxazolidin-3-yl)phenyl]-1H-imidazole-4,5-dicarboxamide methanesulphonate

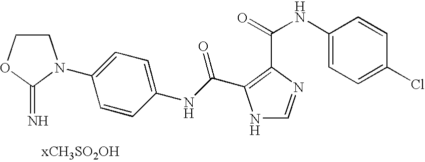

At RT, a total of 6 μl (0.10 mmol) of methanesulphonic acid are added to a solution of 20 mg (0.04 mmol) of N⁵-{4-[(2-{[tert-butyl(dimethyl)silyl]oxy}ethyl)(cyano)-amino]phenyl}-N⁴-(4-chlorophenyl)-1H-imidazole-4,5-dicarboxamide in 10 ml of acetonitrile, and the mixture is stirred at RT for 2 d. The reaction mixture is concentrated under reduced pressure and the residue is triturated with diisopropyl ether, filtered off and dried under reduced pressure.

Yield: 16 mg (77% of theory)

LC-MS (method 3): Rₜ=1.61 min;

MS (ESIpos): m/z=424 [M+H]⁺ (free base);

¹H NMR (400 MHz, DMSO-d₆): δ=12.06 (s, 1H), 11.13 (s, 1H), 9.60 (br. s, 1H), 8.83 (br. s, 1H), 8.10 (s, 1H), 7.97 (d, 2H), 7.84 (d, 2H), 7.57 (d, 2H), 7.48 (d, 2H), 4.87 (t, 1H), 4.26 (t, 2H), 2.34 (s, 3H).

### Example 16

### N-(4-Chlorophenyl)-N′-[4-(2-imino-1,3-oxazolidin-3-yl)phenyl]-1-methyl-1H-pyrrole-3,4-dicarboxamide methanesulphonate

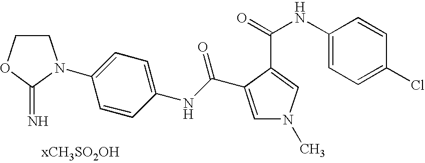

### Step a): N-{4-[(2-{[tert-Butyl(dimethyl)silyl]oxy}ethyl)amino]phenyl}-N′-(4-chlorophenyl)-1-methyl-1H-pyrrole-3,4-dicarboxamide

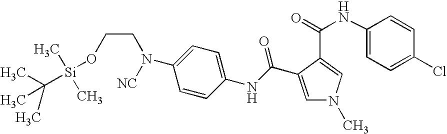

According to the General Method 1, 205 mg (0.74 mmol) of the compound from Example 12A are reacted with 196 mg (0.74 mmol) of the compound from Example 1A.

Yield: 221 mg (57% of theory)

LC-MS (method 1): Rₜ=3.12 min;

MS (ESIpos): m/z=527 [M+H]⁺;

¹H NMR (400 MHz, DMSO-d₆): δ=12.11 (s, 1H), 10.51 (s, 1H), 7.68 (d, 2H), 7.64 (d, 2H), 7.38 (d, 2H), 7.34 (d, 2H), 6.58 (d, 2H), 5.39 (t, 1H), 3.72 (s, 3H), 3.70 (t, 2H), 3.13 (qd, 2H), 0.88 (s, 9H), 0.04 (s, 6H).

### Step b): N-{4-[(2-{[tert-Butyl(dimethyl)silyl]oxy}ethyl)(cyano)amino]phenyl}-N′-(4-chlorophenyl)-1-methyl-1H-pyrrole-3,4-dicarboxamide

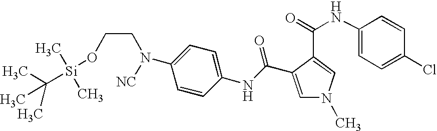

At RT, 72 mg (0.85 mmol) of sodium bicarbonate and 114 μl (0.34 mmol) of a 3 M solution of cyanogen bromide in dichloromethane are added to a solution of 150 mg (0.29 mmol) of N-{4-[(2-{[tert-butyl(dimethyl)silyl]oxy}ethyl)amino]phenyl}-N′-(4-chlorophenyl)-1-methyl-1H-pyrrole-3,4-dicarboxamide in 5 ml of THF. The suspension is stirred at 40° C. for 1 d and then diluted with 4 ml of water and 6 ml of dichloromethane. After phase separation, the organic phase is washed with saturated aqueous sodium bicarbonate solution, dried over sodium sulphate, filtered and concentrated under reduced pressure. The residue is triturated with diethyl ether, filtered off and dried under reduced pressure.

Yield: 108 mg (69% of theory)

LC-MS (method 3): Rₜ=3.23 min;

MS (ESIpos): m/z=552 [M+H]⁺.

### Step c): N-(4-Chlorophenyl)-N′-[4-(2-imino-1,3-oxazolidin-3-yl)phenyl]-1-methyl-1H-pyrrole-3,4-dicarboxamide methanesulphonate

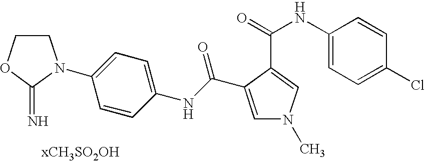

At RT, a total of 27 μl (0.41 mmol) of methanesulphonic acid are added to a solution of 108 mg (0.20 mmol) of N-{4-[(2-{[tert-butyl(dimethyl)silyl]oxy}ethyl)(cyano)-amino]phenyl}-N′-(4-chlorophenyl)-1-methyl-1H-pyrrole-3,4-dicarboxamide in 10 ml of acetonitrile, and the mixture is stirred at RT overnight. The precipitate formed is filtered off and dried under reduced pressure.

Yield: 26 mg (25% of theory)

LC-MS (method 3): Rₜ=1.65 min;

MS (ESIpos): m/z=437 [M+H]⁺ (free base);

¹H NMR (400 MHz, DMSO-d₆): δ=11.67 (s, 1H), 11.29 (s, 1H), 9.55 (br. s, 1H), 8.78 (br. s, 1H), 7.85 (d, 2H), 7.75 (d, 2H), 7.72 (d, 2H), 7.51 (d, 2H), 7.42 (d, 2H), 4.85 (t, 2H), 4.24 (t, 2H), 3.78 (s, 3H), 2.31 (s, 3H).

### Example 17

### N-(5-Chloropyridin-2-yl)-N′-{4-[(2Z)-2-(hydroxyimino)-1,3-oxazolidin-3-yl]-phenyl}pyrazine-2,3-dicarboxamide

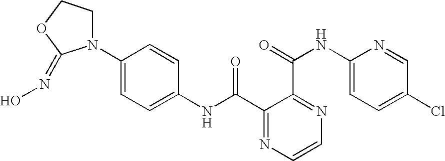

1000 mg (1.87 mmol) of the compound from Example 1, 2596 mg (30.90 mmol, 16.5 eq.) of sodium bicarbonate and 1952 mg (28.09 mmol, 15 eq.) of hydroxylammonium chloride are suspended in 45 ml of an ethanol/water mixture (2:1) and stirred at 60° C. for 14 h. The ethanol is removed under reduced pressure and the solid is separated off by filtration. The latter is purified by RP-HPLC. The resulting crude product is triturated with diethyl ether and filtered, and the solid is dried under high vacuum.

Yield: 125 mg (15% of theory)

HPLC (method 7): Rₜ=3.65 min;

MS (ESIpos): m/z=454 [M+H]⁺;

¹H NMR (400 MHz, DMSO-d₆): δ=11.12 (s, 1H), 10.70 (s, 1H), 8.93-8.91 (s, 2H), 8.60 (s, 1H), 8.42 (s, 1H), 8.24 (d, 1H), 7.99 (d, 1H), 7.74 (d, 2H), 7.51 (d, 2H), 4.44 (t, 2H), 3.94 (t, 2H).

### B. EVALUATION OF THE PHARMACOLOGICAL ACTIVITY

The compounds according to the invention act in particular as selective inhibitors of blood coagulation factor Xa and do not, or only at significantly higher concentrations, inhibit other serine proteases, such as plasmin or trypsin.

Inhibitors of blood coagulation factor Xa are referred to as being “selective” if the IC₅₀ values for factor Xa inhibition are smaller by a factor of at least 100 compared with the IC₅₀ values for the inhibition of other serine proteases, in particular plasmin and trypsin, where, with a view to the test methods for selectivity, reference is made to the test methods described below of Examples B.a.1) and B.a.2).

The advantageous pharmacological properties of the compounds according to the invention can be determined by the following methods:

### a) Test Description (In Vitro)

### a.1) Determination of the Factor Xa Inhibition:

The enzymatic activity of human factor Xa (FXa) is measured using the conversion of a chromogenic substrate specific for FXa. Factor Xa cleaves p-nitroaniline from the chromogenic substrate. The determinations are carried out in microtitre plates as follows:

The test substances, in various concentrations, are dissolved in DMSO and incubated for 10 minutes at 25° C. with human FXa (0.5 mmol/l dissolved in 50 mmol/l of Tris buffer [C,C,C-tris(hydroxymethyl)aminomethane], 150 mmol/l of NaCl, 0.1% BSA [bovine serum albumin], pH=8.3). Pure DMSO is used as control. The chromogenic substrate (150 μmol/l Pefachrome® FXa from Pentapharm) is then added. After an incubation time of 20 minutes at 25° C., the extinction at 405 nm is determined. The extinctions of the text mixtures containing the test substance are compared with the control mixtures without test substance, and the IC₅₀ values are calculated from these data.

Representative activity data from this test are shown in Table 1 below:

TABLE 1

| Example No. | IC₅₀ [nM] |
|---|---|
| 1 | 0.64 |
| 5 | 2.6 |
| 12 | 0.3 |
| 13 | 4.4 |

### a.2) Determination of the Selectivity:

To assess selective FXa inhibition, the test substances are examined for their inhibition of other human serine proteases such as trypsin and plasmin. To determine the enzymatic activity of trypsin (500 mU/ml) and plasmin (3.2 nmol/l), these enzymes are dissolved in Tris buffer (100 mmol/l, 20 mmol/l CaCl₂, pH=8.0) and incubated with test substance or solvent for 10 minutes. The enzymatic reaction is then started by adding the corresponding specific chromogenic substrates (Chromozym Trypsin® and Chromozym Plasmin®; from Roche Diagnostics) and the extinction at 405 nm is determined after 20 minutes. All determinations are carried out at 37° C. The extinctions of the test mixtures containing test substance are compared with the control samples without test substance, and the IC₅₀ values are calculated from these data.

### a.3) Determination of the Anticoagulant Action:

The anticoagulant action of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. Concentration of test substance which effects a doubling of the prothrombin time is determined.

### b) Determination of the Antithrombotic Activity (In Vivo)

### b.1) Arteriovenous Shunt Model (Rabbit):

Fasting rabbits (strain: Esd: NZW) are anaesthetized by intramuscular administration of Rompun/Ketavet solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### C. WORKING EXAMPLES OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. Stirring is continued until the compound according to the invention has dissolved completely.

i.v. Solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. Compound A compound of the formula (I) 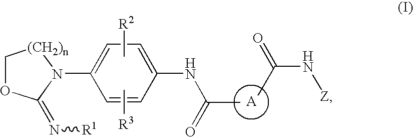
in which
n represents the number 1, 2 or 3,
R¹ represents hydrogen, (C₁-C₄)-alkyl, (C₁-C₄)-alkanoyl, cyano or hydroxyl,
R² and R³ are identical or different and independently of one another represent hydrogen, fluorine, chlorine, cyano, (C₁-C₃)-alkyl, cyclopropyl, trifluoromethyl, hydroxyl, (C₁-C₃)-alkoxy, trifluoromethoxy or amino,
A represents a phenylene or 5- or 6-membered heteroarylene ring where the two carboxamide groupings —CO—NH-phenyl and —CO—NH-Z are located at adjacent ring atoms of the phenylene or heteroarylene ring and phenylene and heteroarylene may additionally be substituted by halogen and/or (C₁-C₄)-alkyl,
and
z represents phenyl, pyridyl, pyrimidinyl, pyrazinyl or thienyl, each of which may be mono- or disubstituted by identical or different substituents selected from the group consisting of fluorine, chlorine, cyano, (C₁-C₄)-alkyl (which for its part may be substituted by amino), ethynyl and amino,
or a salt, solvate, or solvate of a salt thereof.

2. The compound of the formula (I) according to claim 1 in which
A represents a group of the formula 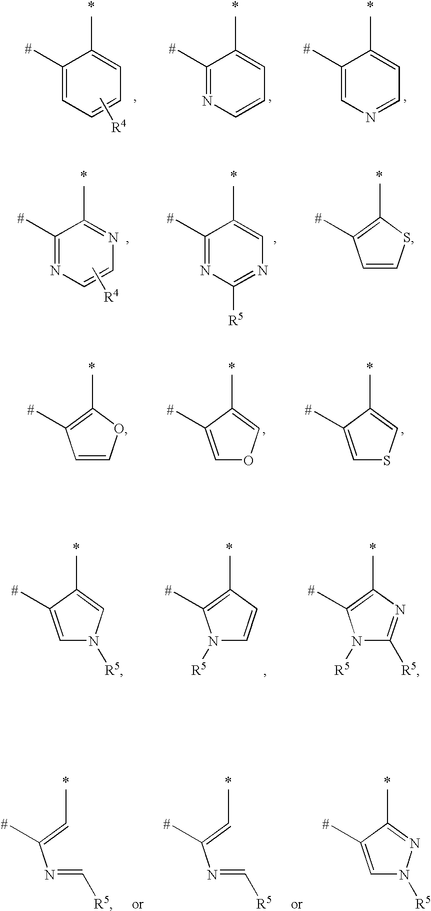
in which
R⁴ represents hydrogen, halogen or (C₁-C₄)-alkyl,
R⁵ represents hydrogen or (C₁-C₄)-alkyl
and
# and * represent the point of attachments to the —CO—NH-phenyl and the —CO—NH-Z grouping.

3. The compound of the formula (I) according to claim 1 or claim 2 in which
Z represents a group of the formula 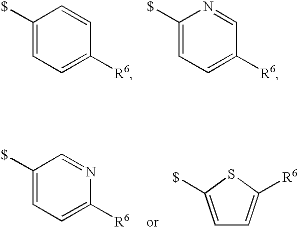
in which
R⁶ represents fluorine, chlorine, cyano, methyl or ethynyl,
and
$ represents the point of attachment to the nitrogen atom.

4. The compound of the formula (I) according to claim 1 in which
n represents the number 1 or 2,
R¹ represents hydrogen,
R² represents hydrogen,
R³ represents hydrogen, fluorine or methyl,
A represents a group of the formula 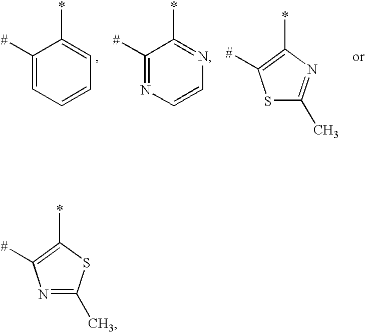
in which
# represents the point of attachment to the —CO—NH-phenyl grouping and
* represents the point of attachment to the —CO—NH-Z grouping,
and
Z represents a group of the formula 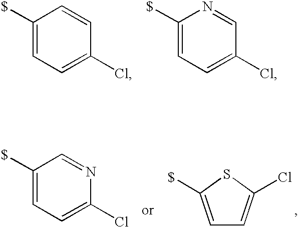
in which $ represents the point of attachment to the nitrogen atom,
or a salt, solvate, or solvate of a salt thereof.

5. The compound of the formula (I) as defined in claim 1 of the following structure: 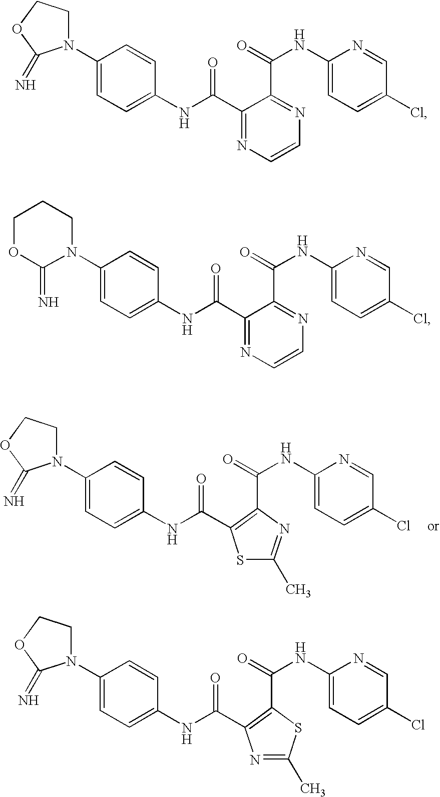
or a salt, solvate, or solvate of a salt thereof.

6. A process for preparing compounds of the formula (I) as defined in claim 1 in which R¹ represents hydrogen,
characterized in that a compound of the formula (II) 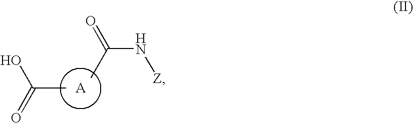
in which A and Z are as defined in claim 1
is initially reacted with a compound of the formula (III) 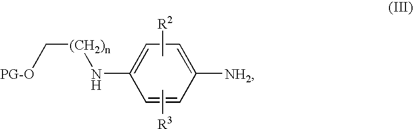
in which n, R² and R³ are as defined in claim 1
and
PG represents a hydroxyl protective group
to give a compound of the formula (IV) 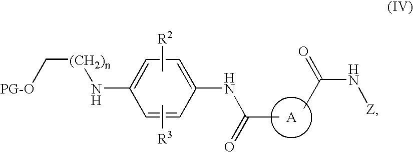
in which n, A, PG, Z, R² and R³ are as defined in claim 1,
then either
(A) converted by removal of the protective group PG into a compound of the formula (V) 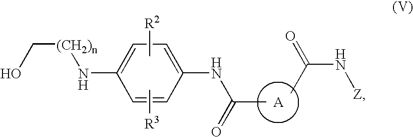
in which n, A, Z, R² and R³ are as defined in claim 1,
and the compound of the formula (V) is then converted with cyanogen bromide into a compound of the formula (I-A) 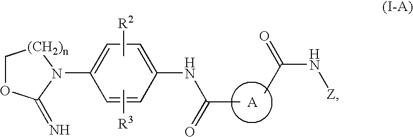
in which n, A, Z, R² and R³ are as defined in claim 1,
or
(B) initially reacted with cyanogen bromide to give a compound of the formula (VI) 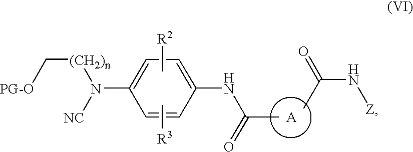
in which n, A, PG, Z, R² and R³ are as defined in claim 1,
then converted by removal of the protective group PG into compounds a compound of the formula (VII) 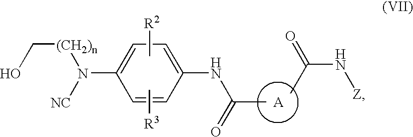
in which n, A, Z, R² and R³ are as defined in claim 1,
and the compound of the formula (VII) is then cyclized to a compound of the formula (I-A)
and the compounds compound of the formula (I-A) is, if appropriate, converted with the appropriate (i) solvents and/or (ii) bases or acids into a solvate, salt, or solvate of a salt.

7. (canceled)

8. (canceled)

9. (canceled)

10. A pharmaceutical composition comprising a compound of the formula (I) as defined in claim 1 in combination with an inert non-toxic pharmaceutically acceptable auxiliary.

11. Medicament, A pharmaceutical composition comprising a compound of the formula (I) as defined in claim 1 in combination with a further active compound other than a compound of formula (I).

12. (canceled)

13. A method for the treatment and/or prophylaxis of thromboembolic disorders in humans and animals which comprises administering an anticoagulatory effective amount of at least one compound of the formula (I) as defined in claim 1 or a medicament as defined in any of claims 10 to 12.

14. A method for preventing blood coagulation in vitro, characterized in that an anticoagulatory effective amount of a compound of the formula (I) as defined in claim 1 is added to said blood.
